SEQUENCE LISTING (208 sequences)

SEQ ID NO: 1 - protein, 20 aa, Artificial Sequence
  synthetic protein fragment
MDFGALPPEVNSVRMYAGPG

SEQ ID NO: 2 - protein, 20 aa, Artificial Sequence
  synthetic protein fragment
EVNSVRMYAGPGSAPMVAAA

SEQ ID NO: 3 - protein, 20 aa, Artificial Sequence
  synthetic protein fragment
AGPGSAPMVAAASAWNGLAA

SEQ ID NO: 4 - protein, 20 aa, Artificial Sequence
  synthetic protein fragment
VAAASAWNGLAAELSSAATG

SEQ ID NO: 5 - protein, 20 aa, Artificial Sequence
  synthetic protein fragment
GLAAELSSAATGYETVITQL

SEQ ID NO: 6 - protein, 20 aa, Artificial Sequence
  synthetic protein fragment
AATGYETVITQLSSEGWLGP

SEQ ID NO: 7 - protein, 20 aa, Artificial Sequence
  synthetic protein fragment
ITQLSSEGWLGPASAAMAEA

SEQ ID NO: 8 - protein, 20 aa, Artificial Sequence
  synthetic protein fragment
WLGPASAAMAEAVAPYVAWM

SEQ ID NO: 9 - protein, 20 aa, Artificial Sequence
  synthetic protein fragment
MAEAVAPYVAWMSAAAAQAE

SEQ ID NO: 10 - protein, 20 aa, Artificial Sequence
  synthetic protein fragment
VAWMSAAAAQAEQAATQARA

SEQ ID NO: 11 - protein, 20 aa, Artificial Sequence
  synthetic protein fragment
AQAEQAATQARAAAAAFEAA

SEQ ID NO: 12 - protein, 20 aa, Artificial Sequence
  synthetic protein fragment
QARAAAAAFEAAFAATVPPP

SEQ ID NO: 13 - protein, 20 aa, Artificial Sequence
  synthetic protein fragment
FEAAFAATVPPPLIAANRAS

SEQ ID NO: 14 - protein, 20 aa, Artificial Sequence
  synthetic protein fragment
VPPPLIAANRASLMQLISTN

SEQ ID NO: 15 - protein, 20 aa, Artificial Sequence
  synthetic protein fragment
NRASLMQLISTNVFGQNTSA

SEQ ID NO: 16 - protein, 20 aa, Artificial Sequence
  synthetic protein fragment
ISTNVFGQNTSAIAAAEAQY

SEQ ID NO: 17 - protein, 20 aa, Artificial Sequence
  synthetic protein fragment
NTSAIAAAEAQYGEMWAQDS

SEQ ID NO: 18 - protein, 20 aa, Artificial Sequence
  synthetic protein fragment
EAQYGEMWAQDSAAMYAYAG

SEQ ID NO: 19 - protein, 20 aa, Artificial Sequence
  synthetic protein fragment
AQDSAAMYAYAGSSASASAV

SEQ ID NO: 20 - protein, 20 aa, Artificial Sequence
  synthetic protein fragment
AYAGSSASASAVTPFSTPPQ

SEQ ID NO: 21 - protein, 20 aa, Artificial Sequence
  synthetic protein fragment
ASAVTPFSTPPQIANPTAQG

SEQ ID NO: 22 - protein, 20 aa, Artificial Sequence
  synthetic protein fragment
TPPQIANPTAQGTQAAAVAT

SEQ ID NO: 23 - protein, 20 aa, Artificial Sequence
  synthetic protein fragment
TAQGTQAAAVATAAGTAQST

SEQ ID NO: 24 - protein, 20 aa, Artificial Sequence
  synthetic protein fragment
AVATAAGTAQSTLTEMITGL

SEQ ID NO: 25 - protein, 20 aa, Artificial Sequence
  synthetic protein fragment
AQSTLTEMITGLPNALQSLT

SEQ ID NO: 26 - protein, 20 aa, Artificial Sequence
  synthetic protein fragment
ITGLPNALQSLTSPLLQSSN

SEQ ID NO: 27 - protein, 20 aa, Artificial Sequence
  synthetic protein fragment
QSLTSPLLQSSNGPLSWLWQ

SEQ ID NO: 28 - protein, 20 aa, Artificial Sequence
  synthetic protein fragment
QSSNGPLSWLWQILFGTPNF

SEQ ID NO: 29 - protein, 20 aa, Artificial Sequence
  synthetic protein fragment
WLWQILFGTPNFPTSISALL

SEQ ID NO: 30 - protein, 20 aa, Artificial Sequence
  synthetic protein fragment
TPNFPTSISALLTDLQPYAS

SEQ ID NO: 31 - protein, 20 aa, Artificial Sequence
  synthetic protein fragment
SALLTDLQPYASFFYNTEGL

SEQ ID NO: 32 - protein, 20 aa, Artificial Sequence
  synthetic protein fragment
PYASFFYNTEGLPYFSIGMG

SEQ ID NO: 33 - protein, 20 aa, Artificial Sequence
  synthetic protein fragment
TEGLPYFSIGMGNNFIQAAK

SEQ ID NO: 34 - protein, 20 aa, Artificial Sequence
  synthetic protein fragment
IGMGNNFIQAAKTLGLIGSA

SEQ ID NO: 35 - protein, 20 aa, Artificial Sequence
  synthetic protein fragment
QAAKTLGLIGSAAPAAVAAA

SEQ ID NO: 36 - protein, 20 aa, Artificial Sequence
  synthetic protein fragment
IGSAAPAAVAAAGDAAKGLP

SEQ ID NO: 37 - protein, 20 aa, Artificial Sequence
  synthetic protein fragment
VAAAGDAAKGLPGLGGMLGG

SEQ ID NO: 38 - protein, 20 aa, Artificial Sequence
  synthetic protein fragment
KGLPGLGGMLGGGPVAAGLG

SEQ ID NO: 39 - protein, 20 aa, Artificial Sequence
  synthetic protein fragment
MLGGGPVAAGLGNAASVGKL

SEQ ID NO: 40 - protein, 20 aa, Artificial Sequence
  synthetic protein fragment
AGLGNAASVGKLSVPPVWSG

SEQ ID NO: 41 - protein, 20 aa, Artificial Sequence
  synthetic protein fragment
VGKLSVPPVWSGPLPGSVTP

SEQ ID NO: 42 - protein, 20 aa, Artificial Sequence
  synthetic protein fragment
VWSGPLPGSVTPGAAPLPVS

SEQ ID NO: 43 - protein, 20 aa, Artificial Sequence
  synthetic protein fragment
SVTPGAAPLPVSTVSAAPEA

SEQ ID NO: 44 - protein, 20 aa, Artificial Sequence
  synthetic protein fragment
LPVSTVSAAPEAAPGSLLGG

SEQ ID NO: 45 - protein, 20 aa, Artificial Sequence
  synthetic protein fragment
APEAAPGSLLGGLPLAGAGG

SEQ ID NO: 46 - protein, 20 aa, Artificial Sequence
  synthetic protein fragment
LLGGLPLAGAGGAGAGPRYG

SEQ ID NO: 47 - protein, 20 aa, Artificial Sequence
  synthetic protein fragment
GAGGAGAGPRYGFRPTVMAR

SEQ ID NO: 48 - protein, 20 aa, Artificial Sequence
  synthetic protein fragment
GAGPRYGFRPTVMARPPFAG

SEQ ID NO: 49 - protein, 20 aa, Artificial Sequence
  synthetic protein fragment
VVDFGALPPEINSARMYAGP

SEQ ID NO: 50 - protein, 20 aa, Artificial Sequence
  synthetic protein fragment
PEINSARMYAGPGSASLVAA

SEQ ID NO: 51 - protein, 20 aa, Artificial Sequence
  synthetic protein fragment
YAGPGSASLVAAAKMWDSVA

SEQ ID NO: 52 - protein, 20 aa, Artificial Sequence
  synthetic protein fragment
LVAAAKMWDSVASDLFSAAS

SEQ ID NO: 53 - protein, 20 aa, Artificial Sequence
  synthetic protein fragment
DSVASDLFSAASAFQSVVWG

SEQ ID NO: 54 - protein, 20 aa, Artificial Sequence
  synthetic protein fragment
SAASAFQSVVWGLTVGSWIG

SEQ ID NO: 55 - protein, 20 aa, Artificial Sequence
  synthetic protein fragment
VVWGLTVGSWIGSSAGLMAA

SEQ ID NO: 56 - protein, 20 aa, Artificial Sequence
  synthetic protein fragment
SWIGSSAGLMAAAASPYVAW

SEQ ID NO: 57 - protein, 20 aa, Artificial Sequence
  synthetic protein fragment
LMAAAASPYVAWMSVTAGQA

SEQ ID NO: 58 - protein, 20 aa, Artificial Sequence
  synthetic protein fragment
YVAWMSVTAGQAQLTAAQVR

SEQ ID NO: 59 - protein, 20 aa, Artificial Sequence
  synthetic protein fragment
AGQAQLTAAQVRVAAAAYET

SEQ ID NO: 60 - protein, 20 aa, Artificial Sequence
  synthetic protein fragment
AQVRVAAAAYETAYRLTVPP

SEQ ID NO: 61 - protein, 20 aa, Artificial Sequence
  synthetic protein fragment
AYETAYRLTVPPPVIAENRT

SEQ ID NO: 62 - protein, 20 aa, Artificial Sequence
  synthetic protein fragment
TVPPPVIAENRTELMTLTAT

SEQ ID NO: 63 - protein, 20 aa, Artificial Sequence
  synthetic protein fragment
ENRTELMTLTATNLLGQNTP

SEQ ID NO: 64 - protein, 20 aa, Artificial Sequence
  synthetic protein fragment
LTATNLLGQNTPAIEANQAA

SEQ ID NO: 65 - protein, 20 aa, Artificial Sequence
  synthetic protein fragment
QNTPAIEANQAAYSQMWGQD

SEQ ID NO: 66 - protein, 20 aa, Artificial Sequence
  synthetic protein fragment
NQAAYSQMWGQDAEAMYGYA

SEQ ID NO: 67 - protein, 20 aa, Artificial Sequence
  synthetic protein fragment
WGQDAEAMYGYAATAATATE

SEQ ID NO: 68 - protein, 20 aa, Artificial Sequence
  synthetic protein fragment
YGYAATAATATEALLPFEDA

SEQ ID NO: 69 - protein, 20 aa, Artificial Sequence
  synthetic protein fragment
TATEALLPFEDAPLITNPGG

SEQ ID NO: 70 - protein, 20 aa, Artificial Sequence
  synthetic protein fragment
FEDAPLITNPGGLLEQAVAV

SEQ ID NO: 71 - protein, 20 aa, Artificial Sequence
  synthetic protein fragment
NPGGLLEQAVAVEEAIDTAA

SEQ ID NO: 72 - protein, 20 aa, Artificial Sequence
  synthetic protein fragment
AVAVEEAIDTAAANQLMNNV

SEQ ID NO: 73 - protein, 20 aa, Artificial Sequence
  synthetic protein fragment
DTAAANQLMNNVPQALQQLA

SEQ ID NO: 74 - protein, 20 aa, Artificial Sequence
  synthetic protein fragment
MNNVPQALQQLAQPAQGVVP

SEQ ID NO: 75 - protein, 20 aa, Artificial Sequence
  synthetic protein fragment
QQLAQPAQGVVPSSKLGGLW

SEQ ID NO: 76 - protein, 20 aa, Artificial Sequence
  synthetic protein fragment
GVVPSSKLGGLWTAVSPHLS

SEQ ID NO: 77 - protein, 20 aa, Artificial Sequence
  synthetic protein fragment
GGLWTAVSPHLSPLSNVSSI

SEQ ID NO: 78 - protein, 20 aa, Artificial Sequence
  synthetic protein fragment
PHLSPLSNVSSIANNHMSMM

SEQ ID NO: 79 - protein, 20 aa, Artificial Sequence
  synthetic protein fragment
VSSIANNHMSMMGTGVSMTN

SEQ ID NO: 80 - protein, 20 aa, Artificial Sequence
  synthetic protein fragment
MSMMGTGVSMTNTLHSMLKG

SEQ ID NO: 81 - protein, 20 aa, Artificial Sequence
  synthetic protein fragment
SMTNTLHSMLKGLAPAAAQA

SEQ ID NO: 82 - protein, 20 aa, Artificial Sequence
  synthetic protein fragment
MLKGLAPAAAQAVETAAENG

SEQ ID NO: 83 - protein, 20 aa, Artificial Sequence
  synthetic protein fragment
AAQAVETAAENGVWAMSSLG

SEQ ID NO: 84 - protein, 20 aa, Artificial Sequence
  synthetic protein fragment
AENGVWAMSSLGSQLGSSLG

SEQ ID NO: 85 - protein, 20 aa, Artificial Sequence
  synthetic protein fragment
SSLGSQLGSSLGSSGLGAGV

SEQ ID NO: 86 - protein, 20 aa, Artificial Sequence
  synthetic protein fragment
SSLGSSGLGAGVAANLGRAA

SEQ ID NO: 87 - protein, 20 aa, Artificial Sequence
  synthetic protein fragment
GAGVAANLGRAASVGSLSVP

SEQ ID NO: 88 - protein, 20 aa, Artificial Sequence
  synthetic protein fragment
GRAASVGSLSVPPAWAAANQ

SEQ ID NO: 89 - protein, 20 aa, Artificial Sequence
  synthetic protein fragment
LSVPPAWAAANQAVTPAARA

SEQ ID NO: 90 - protein, 20 aa, Artificial Sequence
  synthetic protein fragment
AANQAVTPAARALPLTSLTS

SEQ ID NO: 91 - protein, 20 aa, Artificial Sequence
  synthetic protein fragment
AARALPLTSLTSAAQTAPGH

SEQ ID NO: 92 - protein, 20 aa, Artificial Sequence
  synthetic protein fragment
SLTSAAQTAPGHMLGGLPLG

SEQ ID NO: 93 - protein, 20 aa, Artificial Sequence
  synthetic protein fragment
APGHMLGGLPLGHSVNAGSG

SEQ ID NO: 94 - protein, 20 aa, Artificial Sequence
  synthetic protein fragment
LPLGHSVNAGSGINNALRVP

SEQ ID NO: 95 - protein, 20 aa, Artificial Sequence
  synthetic protein fragment
AGSGINNALRVPARAYAIPR

SEQ ID NO: 96 - protein, 20 aa, Artificial Sequence
  synthetic protein fragment
NNALRVPARAYAIPRTPAAG

SEQ ID NO: 97 - protein, 20 aa, Artificial Sequence
  synthetic protein fragment
MSRAFIIDPTISAIDGLYDL

SEQ ID NO: 98 - protein, 20 aa, Artificial Sequence
  synthetic protein fragment
PTISAIDGLYDLLGIGIPNQ

SEQ ID NO: 99 - protein, 20 aa, Artificial Sequence
  synthetic protein fragment
LYDLLGIGIPNQGGILYSSL

SEQ ID NO: 100 - protein, 20 aa, Artificial Sequence
  synthetic protein fragment
IPNQGGILYSSLEYFEKALE

SEQ ID NO: 101 - protein, 20 aa, Artificial Sequence
  synthetic protein fragment
YSSLEYFEKALEELAAAFPG

SEQ ID NO: 102 - protein, 20 aa, Artificial Sequence
  synthetic protein fragment
KALEELAAAFPGDGWLGSAA

SEQ ID NO: 103 - protein, 20 aa, Artificial Sequence
  synthetic protein fragment
AFPGDGWLGSAADKYAGKNR

SEQ ID NO: 104 - protein, 20 aa, Artificial Sequence
  synthetic protein fragment
GSAADKYAGKNRNHVNFFQE

SEQ ID NO: 105 - protein, 20 aa, Artificial Sequence
  synthetic protein fragment
GKNRNHVNFFQELADLDRQL

SEQ ID NO: 106 - protein, 20 aa, Artificial Sequence
  synthetic protein fragment
FFQELADLDRQLISLIHDQA

SEQ ID NO: 107 - protein, 20 aa, Artificial Sequence
  synthetic protein fragment
DRQLISLIHDQANAVQTTRD

SEQ ID NO: 108 - protein, 20 aa, Artificial Sequence
  synthetic protein fragment
HDQANAVQTTRDILEGAKKG

SEQ ID NO: 109 - protein, 20 aa, Artificial Sequence
  synthetic protein fragment
TTRDILEGAKKGLEFVRPVA

SEQ ID NO: 110 - protein, 20 aa, Artificial Sequence
  synthetic protein fragment
AKKGLEFVRPVAVDLTYIPV

SEQ ID NO: 111 - protein, 20 aa, Artificial Sequence
  synthetic protein fragment
RPVAVDLTYIPVVGHALSAA

SEQ ID NO: 112 - protein, 20 aa, Artificial Sequence
  synthetic protein fragment
YIPVVGHALSAAFQAPFCAG

SEQ ID NO: 113 - protein, 20 aa, Artificial Sequence
  synthetic protein fragment
LSAAFQAPFCAGAMAVVGGA

SEQ ID NO: 114 - protein, 20 aa, Artificial Sequence
  synthetic protein fragment
FCAGAMAVVGGALAYLAVKT

SEQ ID NO: 115 - protein, 20 aa, Artificial Sequence
  synthetic protein fragment
VGGALAYLAVKTLINATQLL

SEQ ID NO: 116 - protein, 20 aa, Artificial Sequence
  synthetic protein fragment
AVKTLINATQLLKLLAKLAE

SEQ ID NO: 117 - protein, 20 aa, Artificial Sequence
  synthetic protein fragment
TQLLKLLAKLAELVAAAIAD

SEQ ID NO: 118 - protein, 20 aa, Artificial Sequence
  synthetic protein fragment
KLAELVAAAIADIISDVADI

SEQ ID NO: 119 - protein, 20 aa, Artificial Sequence
  synthetic protein fragment
AIADIISDVADIIKGILGEV

SEQ ID NO: 120 - protein, 20 aa, Artificial Sequence
  synthetic protein fragment
VADIIKGILGEVWEFITNAL

SEQ ID NO: 121 - protein, 20 aa, Artificial Sequence
  synthetic protein fragment
LGEVWEFITNALNGLKELWD

SEQ ID NO: 122 - protein, 20 aa, Artificial Sequence
  synthetic protein fragment
TNALNGLKELWDKLTGWVTG

SEQ ID NO: 123 - protein, 20 aa, Artificial Sequence
  synthetic protein fragment
ELWDKLTGWVTGLFSRGWSN

SEQ ID NO: 124 - protein, 20 aa, Artificial Sequence
  synthetic protein fragment
WVTGLFSRGWSNLESFFAGV

SEQ ID NO: 125 - protein, 20 aa, Artificial Sequence
  synthetic protein fragment
GWSNLESFFAGVPGLTGATS

SEQ ID NO: 126 - protein, 20 aa, Artificial Sequence
  synthetic protein fragment
FAGVPGLTGATSGLSQVTGL

SEQ ID NO: 127 - protein, 20 aa, Artificial Sequence
  synthetic protein fragment
GATSGLSQVTGLFGAAGLSA

SEQ ID NO: 128 - protein, 20 aa, Artificial Sequence
  synthetic protein fragment
VTGLFGAAGLSASSGLAHAD

SEQ ID NO: 129 - protein, 20 aa, Artificial Sequence
  synthetic protein fragment
GLSASSGLAHADSLASSASL

SEQ ID NO: 130 - protein, 20 aa, Artificial Sequence
  synthetic protein fragment
AHADSLASSASLPALAGIGG

SEQ ID NO: 131 - protein, 20 aa, Artificial Sequence
  synthetic protein fragment
SASLPALAGIGGGSGFGGLP

SEQ ID NO: 132 - protein, 20 aa, Artificial Sequence
  synthetic protein fragment
GIGGGSGFGGLPSLAQVHAA

SEQ ID NO: 133 - protein, 20 aa, Artificial Sequence
  synthetic protein fragment
GGLPSLAQVHAASTRQALRP

SEQ ID NO: 134 - protein, 20 aa, Artificial Sequence
  synthetic protein fragment
VHAASTRQALRPRADGPVGA

SEQ ID NO: 135 - protein, 20 aa, Artificial Sequence
  synthetic protein fragment
ALRPRADGPVGAAAEQVGGQ

SEQ ID NO: 136 - protein, 20 aa, Artificial Sequence
  synthetic protein fragment
PVGAAAEQVGGQSQLVSAQG

SEQ ID NO: 137 - protein, 20 aa, Artificial Sequence
  synthetic protein fragment
VGGQSQLVSAQGSQGMGGPV

SEQ ID NO: 138 - protein, 20 aa, Artificial Sequence
  synthetic protein fragment
SAQGSQGMGGPVGMGGMHPS

SEQ ID NO: 139 - protein, 20 aa, Artificial Sequence
  synthetic protein fragment
GGPVGMGGMHPSSGASKGTT

SEQ ID NO: 140 - protein, 20 aa, Artificial Sequence
  synthetic protein fragment
MHPSSGASKGTTTKKYSEGA

SEQ ID NO: 141 - protein, 20 aa, Artificial Sequence
  synthetic protein fragment
KGTTTKKYSEGAAAGTEDAE

SEQ ID NO: 142 - protein, 20 aa, Artificial Sequence
  synthetic protein fragment
SEGAAAGTEDAERAPVEADA

SEQ ID NO: 143 - protein, 20 aa, Artificial Sequence
  synthetic protein fragment
EDAERAPVEADAGGGQKVLV

SEQ ID NO: 144 - protein, 16 aa, Artificial Sequence
  synthetic protein fragment
EADAGGGQKVLVRNVV

SEQ ID NO: 145 - protein, 20 aa, Artificial Sequence
  synthetic protein fragment
RAPVEADAGGGQKVLVRNVV

SEQ ID NO: 146 - protein, 20 aa, Artificial Sequence
  synthetic protein fragment
VPNRRRRKLSTAMSAVAALA

SEQ ID NO: 147 - protein, 20 aa, Artificial Sequence
  synthetic protein fragment
LSTAMSAVAALAVASPCAYF

SEQ ID NO: 148 - protein, 20 aa, Artificial Sequence
  synthetic protein fragment
AALAVASPCAYFLVYESTET

SEQ ID NO: 149 - protein, 20 aa, Artificial Sequence
  synthetic protein fragment
CAYFLVYESTETTERPEHHE

SEQ ID NO: 150 - protein, 20 aa, Artificial Sequence
  synthetic protein fragment
STETTERPEHHEFKQAAVLT

SEQ ID NO: 151 - protein, 20 aa, Artificial Sequence
  synthetic protein fragment
EHHEFKQAAVLTDLPGELMS

SEQ ID NO: 152 - protein, 20 aa, Artificial Sequence
  synthetic protein fragment
AVLTDLPGELMSALSQGLSQ

SEQ ID NO: 153 - protein, 20 aa, Artificial Sequence
  synthetic protein fragment
ELMSALSQGLSQFGINIPPV

SEQ ID NO: 154 - protein, 20 aa, Artificial Sequence
  synthetic protein fragment
GLSQFGINIPPVPSLTGSGD

SEQ ID NO: 155 - protein, 20 aa, Artificial Sequence
  synthetic protein fragment
IPPVPSLTGSGDASTGLTGP

SEQ ID NO: 156 - protein, 20 aa, Artificial Sequence
  synthetic protein fragment
GSGDASTGLTGPGLTSPGLT

SEQ ID NO: 157 - protein, 20 aa, Artificial Sequence
  synthetic protein fragment
LTGPGLTSPGLTSPGLTSPG

SEQ ID NO: 158 - protein, 20 aa, Artificial Sequence
  synthetic protein fragment
PGLTSPGLTSPGLTDPALTS

SEQ ID NO: 159 - protein, 20 aa, Artificial Sequence
  synthetic protein fragment
TSPGLTDPALTSPGLTPTLP

SEQ ID NO: 160 - protein, 20 aa, Artificial Sequence
  synthetic protein fragment
ALTSPGLTPTLPGSLAAPGT

SEQ ID NO: 161 - protein, 20 aa, Artificial Sequence
  synthetic protein fragment
PTLPGSLAAPGTTLAPTPGV

SEQ ID NO: 162 - protein, 20 aa, Artificial Sequence
  synthetic protein fragment
APGTTLAPTPGVGANPALTN

SEQ ID NO: 163 - protein, 20 aa, Artificial Sequence
  synthetic protein fragment
TPGVGANPALTNPALTSPTG

SEQ ID NO: 164 - protein, 20 aa, Artificial Sequence
  synthetic protein fragment
ALTNPALTSPTGATPGLTSP

SEQ ID NO: 165 - protein, 20 aa, Artificial Sequence
  synthetic protein fragment
SPTGATPGLTSPTGLDPALG

SEQ ID NO: 166 - protein, 20 aa, Artificial Sequence
  synthetic protein fragment
LTSPTGLDPALGGANEIPIT

SEQ ID NO: 167 - protein, 20 aa, Artificial Sequence
  synthetic protein fragment
PALGGANEIPITTPVGLDPG

SEQ ID NO: 168 - protein, 20 aa, Artificial Sequence
  synthetic protein fragment
IPITTPVGLDPGADGTYPIL

SEQ ID NO: 169 - protein, 20 aa, Artificial Sequence
  synthetic protein fragment
LDPGADGTYPILGDPTLGTI

SEQ ID NO: 170 - protein, 20 aa, Artificial Sequence
  synthetic protein fragment
YPILGDPTLGTIPSSPATTS

SEQ ID NO: 171 - protein, 20 aa, Artificial Sequence
  synthetic protein fragment
LGTIPSSPATTSTGGGGLVN

SEQ ID NO: 172 - protein, 20 aa, Artificial Sequence
  synthetic protein fragment
ATTSTGGGGLVNDVMQVANE

SEQ ID NO: 173 - protein, 20 aa, Artificial Sequence
  synthetic protein fragment
GLVNDVMQVANELGASQAID

SEQ ID NO: 174 - protein, 20 aa, Artificial Sequence
  synthetic protein fragment
VANELGASQAIDLLKGVLMP

SEQ ID NO: 175 - protein, 20 aa, Artificial Sequence
  synthetic protein fragment
QAIDLLKGVLMPSIMQAVQN

SEQ ID NO: 176 - protein, 20 aa, Artificial Sequence
  synthetic protein fragment
VLMPSIMQAVQNGGAAAPAA

SEQ ID NO: 177 - protein, 20 aa, Artificial Sequence
  synthetic protein fragment
AVQNGGAAAPAASPPVPPIP

SEQ ID NO: 178 - protein, 20 aa, Artificial Sequence
  synthetic protein fragment
APAASPPVPPIPAAAAVPPT

SEQ ID NO: 179 - protein, 20 aa, Artificial Sequence
  synthetic protein fragment
PPIPAAAAVPPTDPITVPVA

SEQ ID NO: 180 - protein, 95 aa, Artificial Sequence
  synthetic protein fragment
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 181 - protein, 100 aa, Artificial Sequence
  synthetic protein fragment
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 182 - protein, 103 aa, Artificial Sequence
  synthetic protein fragment
MTENLTVQPERLGVLASHHDNAAVDASSGVEAAAGLGESVAITHGPYCSQFNDTLNVYLTAHNALGSSLHTAGVDLAKSLRIAAKIYSEADEAWRKAIDGLFT

SEQ ID NO: 183 - protein, 393 aa, Artificial Sequence
  synthetic protein fragment
MDFGALPPEVNSVRMYAGPGSAPMVAAASAWNGLAAELSSAATGYETVITQLSSEGWLGPASAAMAEAVAPYVAWMSAAAAQAEQAATQARAAAAAFEAAFAATVPPPLIAANRASLMQLISTNVFGQNTSAIAAAEAQYGEMWAQDSAAMYAYAGSSASASAVTPFSTPPQIANPTAQGTQAAAVATAAGTAQSTLTEMITGLPNALQSLTSPLLQSSNGPLSWLWQILFGTPNFPTSISALLTDLQPYASFFYNTEGLPYFSIGMGNNFIQSAKTLGLIGSAAPAAVAAAGDAAKGLPGLGGMLGGGPVAAGLGNAASVGKLSVPPVWSGPLPGSVTPGAAPLPVSTVSAAPEAAPGSLLGGLPLAGAGGAGAGPRYGFRPTVMARPPFAG

SEQ ID NO: 184 - protein, 97 aa, Artificial Sequence
  synthetic protein fragment
MSLLDAHIPQLIASHTAFAAKAGLMRHTIGQAEQQAMSAQAFHQGESAAAFQGAHARFVAAAAKVNTLLDIAQANLGEAAGTYVAADAAAASSYTGF

SEQ ID NO: 185 - protein, 393 aa, Artificial Sequence
  synthetic protein fragment
VVDFGALPPEINSARMYAGPGSASLVAAAKMWDSVASDLFSAASAFQSVVWGLTVGSWIGSSAGLMAAAASPYVAWMSVTAGQAQLTAAQVRVAAAAYETAYRLTVPPPVIAENRTELMTLTATNLLGQNTPAIEANQAAYSQMWGQDAEAMYGYAATAATATEALLPFEDAPLITNPGGLLEQAVAVEEAIDTAAANQLMNNVPQALQQLAQPAQGVVPSSKLGGLWTAVSPHLSPLSNVSSIANNHMSMMGTGVSMTNTLHSMLKGLAPAAAQAVETAAENGVWAMSSLGSQLGSSLGSSGLGAGVAANLGRAASVGSLSVPPAWAAANQAVTPAARALPLTSLTSAAQTAPGHMLGGLPLGHSVNAGSGINNALRVPARAYAIPRTPAAG

SEQ ID NO: 186 - protein, 284 aa, Artificial Sequence
  synthetic protein fragment
VPNRRRRKLSTAMSAVAALAVASPCAYFLVYESTETTERPEHHEFKQAAVLTDLPGELMSALSQGLSQFGINIPPVPSLTGSGDASTGLTGPGLTSPGLTSPGLTSPGLTDPALTSPGLTPTLPGSLAAPGTTLAPTPGVGANPALTNPALTSPTGATPGLTSPTGLDPALGGANEIPITTPVGLDPGADGTYPILGDPTLGTIPSSPATTSTGGGGLVNDVMQVANELGASQAIDLLKGVLMPSIMQAVQNGGAAAPAASPPVPPIPAAAAVPPTDPITVPVA

SEQ ID NO: 187 - protein, 40 aa, Artificial Sequence
  synthetic protein fragment
MSRAFIIDPTISAIDGLYDLLGIGIPNQGGILYSSLEYFE

SEQ ID NO: 188 - protein, 40 aa, Artificial Sequence
  synthetic protein fragment
LGIGIPNQGGILYSSLEYFEKALEELAAAFPGDGWLGSAA

SEQ ID NO: 189 - protein, 40 aa, Artificial Sequence
  synthetic protein fragment
KALEELAAAFPGDGWLGSAADKYAGKNRNHVNFFQELADL

SEQ ID NO: 190 - protein, 40 aa, Artificial Sequence
  synthetic protein fragment
DKYAGKNRNHVNFFQELADLDRQLISLIHDQANAVQTTRD

SEQ ID NO: 191 - protein, 40 aa, Artificial Sequence
  synthetic protein fragment
DRQLISLIHDQANAVQTTRDILEGAKKGLEFVRPVAVDLT

SEQ ID NO: 192 - protein, 40 aa, Artificial Sequence
  synthetic protein fragment
ILEGAKKGLEFVRPVAVDLTYIPVVGHALSAAFQAPFCAG

SEQ ID NO: 193 - protein, 40 aa, Artificial Sequence
  synthetic protein fragment
YIPVVGHALSAAFQAPFCAGAMAVVGGALAYLVVKTLINA

SEQ ID NO: 194 - protein, 40 aa, Artificial Sequence
  synthetic protein fragment
IISDVADIIKGTLGEVWEFITNALNGLKELWDKLTGWVTG

SEQ ID NO: 195 - protein, 40 aa, Artificial Sequence
  synthetic protein fragment
TNALNGLKELWDKLTGWVTGLFSRGWSNLESFFAGVPGLT

SEQ ID NO: 196 - protein, 40 aa, Artificial Sequence
  synthetic protein fragment
GATSGLSQVTGLFGAAGLSASSGLAHADSLASSASLPALA

SEQ ID NO: 197 - protein, 40 aa, Artificial Sequence
  synthetic protein fragment
SSGLAHADSLASSASLPALAGIGGGSGFGGLPSLAQVHAA

SEQ ID NO: 198 - protein, 40 aa, Artificial Sequence
  synthetic protein fragment
GIGGGSGFGGLPSLAQVHAASTRQALRPRADGPVGAAAEQ

SEQ ID NO: 199 - protein, 40 aa, Artificial Sequence
  synthetic protein fragment
STRQALRPRADGPVGAAAEQVGGQSQLVSAQGSQGMGGPV

SEQ ID NO: 200 - protein, 40 aa, Artificial Sequence
  synthetic protein fragment
VGGQSQLVSAQGSQGMGGPVGMGGMHPSSGASKGTTTKKY

SEQ ID NO: 201 - protein, 40 aa, Artificial Sequence
  synthetic protein fragment
GMGGMHPSSGASKGTTTKKYSEGAAAGTEDAERAPVEADA

SEQ ID NO: 202 - protein, 40 aa, Artificial Sequence
  synthetic protein fragment
KGTTTKKYSEGAAAGTEDAERAPVEADAGGGQKVLVRNVV

SEQ ID NO: 203 - protein, 25 aa, Artificial Sequence
  synthetic protein fragment
AMAVVGGALAYLVVKTLINATQLLK

SEQ ID NO: 204 - protein, 25 aa, Artificial Sequence
  synthetic protein fragment
VKTLINATQLLKLLAKLAELVAAAI

SEQ ID NO: 205 - protein, 25 aa, Artificial Sequence
  synthetic protein fragment
LAKLAELVAAAIADIISDVADIIKG

SEQ ID NO: 206 - protein, 20 aa, Artificial Sequence
  synthetic protein fragment
ESFAGVPGLTGATSGLSQVT

SEQ ID NO: 207 - protein, 329 aa, Artificial Sequence
  synthetic protein fragment
MTMITPSLRRDIHMHHHHHHSMDTENLTVQPERLGVLASHHDNAAVDASSGVEAAAGLGESVAITHGPYCSQFNDTLNVYLTAHNALGSSLHTAGVDLAKSLRIAAKIYSEADEAWRKAIDGLFTHMTEQQWNFAGIEAAASAIQGNVTSIHSLLDEGKQSLTKLAAAWGGSGSEAYQGVQQKWDATATELNNALQNLARTISEAGQAMASTEGNVTGMFAGGMAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFHHHHHH

SEQ ID NO: 208 - protein, 392 aa, Artificial Sequence
  synthetic protein fragment
MSRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVV

(19) United States
(12) Patent Application Publication
(10) Pub. No.: US 2022/0252595 A1
(43) Pub. Date: Aug. 11, 2022

(54) DIAGNOSTIC REAGENT

(71) Applicant: The Secretary of State for Environment, Food and Rural Affairs, Addlestone, Surrey (GB)

(72) Inventors: Gareth JONES, Addlestone (GB); Sonya MIDDLETON, Addlestone, Surrey (GB); Sabine STEINBACH, Addlestone, Surrey (GB); Hans Martin VORDERMEIER, Addlestone, Surrey (GB)

(21) Appl. No.: 17/626,090

(22) PCT Filed: Jul. 9, 2020
(86) PCT No.: PCT/GB2020/051654

§ 371 Date: Jan. 10, 2022

(30) Foreign Application Priority Data
Jul. 11, 2019 (GB) 1909953.0

(51) Int. Cl. G01N 33/569

(52) U.S. Cl. CPC G01N 33/5695

(57) ABSTRACT

The invention provides a Mycobacterium Tuberculosis Complex (MTC) (for example, M. bovis and/or M. tuberculosis) diagnostic reagent comprising the reagent components:

a. a Rv3616c antigen polypeptide and/or a Rv3616c antigenic cocktail;

b. a Rv1789 antigen polypeptide and/or a Rv1789 antigenic cocktail;

c. a Rv3810 antigen polypeptide and/or a Rv3810 antigenic cocktail; and

d. a Rv3478 antigen polypeptide and/or a Rv3478 antigenic cocktail.

The diagnostic reagent may further comprise one or more of a ESAT-6 antigen polypeptide and/or a ESAT-6 antigenic cocktail; a CFP-10 antigen polypeptide and/or a CFP-10 antigenic cocktail; a Rv3615c antigen polypeptide and/or a Rv3615c antigenic cocktail; and/or SEQ ID NO:207 (a fusion protein of ESAT-6 and CFP-10 and Rv3615c). There are also provided methods and kits involving use of the diagnostic reagent.

### TECHNICAL FIELD

This invention relates to reagents for use in a test for detection of mycobacterium infections, particularly Mycobacterium tuberculosis and Mycobacterium bovis, in animals such as cattle.

### BACKGROUND

Tuberculosis, caused by Mycobacterium tuberculosis var tuberculosis, is one of the world's deadliest infectious diseases, claiming as many as three human lives every minute (Corbett et al., (2003) Archives of internal medicine 163, 1009-1021; WHO Global TB Report available at www.who.int/tb/publications/factsheet_global.pdf). The closely related Mycobacterium tuberculosis var bovis (M. bovis) is the main cause of tuberculosis in a wide variety of animal hosts including cattle (bovine TB or bTB), and significantly limits livestock productivity (Gagneux, (2018) Nature Reviews Microbiology 16, 202; Müller et al., (2013) Emerging Infectious Diseases 19, 899-90; Smith et al., (2009) Nature Reviews Microbiology 7, 537). Importantly, bTB represents a serious zoonotic threat, and is estimated to cause approximately 10% of the total human TB cases worldwide (Thoen et al., (2006) Veterinary Microbiology 112, 339-345; Jiang et al., (2015) Scientific Reports 5, 8538; Egbe et al., (2016) Scientific Reports 6, 24320). While bTB is well controlled in most high-income countries through the implementation of strict test and cull strategies, the disease remains endemic in most low- and middle-income countries where national control programs have not yet been implemented for socio-economic reasons, and hence continues to contribute major losses to animal productivity along with human morbidity and mortality (Brooks-Pollock et al., (2014) Nature 511, 228; Dean et al., (2018) The Lancet. Infectious diseases 18, 137-138).

Based on an approach initially established more than a century ago, the current standard for diagnosis of bTB in animals works by measuring cell-mediated immune response following an intradermal skin test with the poorly defined and highly variable tuberculin skin test (TST) antigen (de la Rua-Domenech et al., (2006) Research in Veterinary Science 81, 190-210; Schiller et al., (2010) Transboundary and Emerging Diseases 57, 205-220). More recently, an in vitro interferon-γ release assay (IGRA) has been introduced as an ancillary test in order to improve the overall sensitivity of detection of bTB-infected animals (Wood & Jones, (2001) Tuberculosis (Edinburgh, Scotland) 81, 147-155). The poorly standardized stimulating antigens in the TST (“purified protein derivative” or PPD) are extracts obtained from the heat-killed cultures of specified strains of mycobacteria grown on glycerol broth (Good et al., (2018) Frontiers in Veterinary Science 5, 59; Yang et al., (2012) FEMS immunology and medical microbiology 66, 273-280). For instance, bovine PPD (PPD-B) is derived from an extract of M. bovis AN5 strain culture, while avian PPD (PPD-A) is a similarly prepared extract from M. avium subsp. avium D4ER (OIE. Manual of diagnostic Test and Vaccines for Terrestrial Animals. World Organisation for Animal Health 2019; www.oie.int/standard-setting/terrestrial-manual/access-online/, accessed on 9 Jul. 2019). In regions with high exposure to environmental mycobacteria, the difference in increase in skin induration reaction between bovine and avian PPD (i.e. PPD B-A) is ascertained using the single intradermal comparative cervical tuberculin test (SICCT) to improve test specificity, but this is also known to reduce assay sensitivity (de la Rua-Domenech et al., (2006) Research in Veterinary Science 81, 190-210).

Furthermore, in addition to the poor standardization of the PPDs, the presence of cross-reactive antigens between the pathogenic and vaccine strains in the crude whole cell antigen preparation renders the PPD-based TST unable to differentiate infected from bacille Calmette-Guérin (BCG) vaccinated animals, thereby limiting opportunities for the development of BCG vaccination-based control programs (Waters et al., (2012) Vaccine 30, 2611-2622).

Hence, there is a well-recognized and urgent need to develop defined antigen based bTB diagnostic assays with the ability to ‘differentiate infected from vaccinated animals’ (i.e. “DIVA” assays) for use alongside future (vaccination-based) control programs in regions where conventional test and cull strategies are not feasible for socio-economic reasons (Vordermeier et al., (2009) Transboundary and Emerging Diseases 56, 240-247).

Over the past two decades, comparative genomic and transcriptome analyses have identified several specific M. bovis antigens with DIVA capability, including ESAT-6, CFP-10 and Rv3615c, that are present in field strains of M. bovis but are either absent or not immunogenic in the widely used vaccine strain, BCG (Vordermeier et al., (1999) Clinical and Diagnostic Laboratory Immunology 6, 675-682; Vordermeier et al., (2016) Annu Rev Anim Biosci 4, 87-109). When used in combination, these antigens have shown promise in both detecting infected animals as well as differentiating them from those vaccinated with BCG (Whelan et al., (2010) Journal of Clinical Microbiology 48, 3176-3181).

There remains, however, a need to develop an improved skin test antigen with DIVA capability that might serve as a reliable, easy to produce and fit-for-purpose assay for diagnosis of bTB.

### SUMMARY OF THE INVENTION

The inventors have identified a combination of antigenic proteins or fragments thereof which may be used to complement DIVA skin test (referred to herein as “DST”) antigens, to increase overall signal strength and sensitivity.

Accordingly, a first aspect of the invention provides a Mycobacterium Tuberculosis Complex (MTC) diagnostic reagent comprising the reagent components:
a) a Rv3616c antigen polypeptide and/or a Rv3616c antigenic cocktail;
b) a Rv1789 antigen polypeptide and/or a Rv1789 antigenic cocktail;
c) a Rv3810 antigen polypeptide and/or a Rv3810 antigenic cocktail; and
d) a Rv3478 antigen polypeptide and/or a Rv3478 antigenic cocktail.

The Mycobacterium Tuberculosis Complex (MTC) is a genetically related group of Mycobacterium species that are capable of causing tuberculosis. The MTC includes Mycobacterium tuberculosis (M. tuberculosis), Mycobacterium africanum (M. africanum), Mycobacterium orygis (M. orygis, which may otherwise be referred to as the oryx bacilli), Mycobacterium bovis (M. bovis), Mycobacterium microti (M. microti), Mycobacterium canetti (M. canetti), Mycobacterium caprae (M. caprae), Mycobacterium pinnipedii (M. pinnipedii), Mycobacterium suricattae (M. suricattae) and Mycobacterium mungi (M. mungi). Many of the sequences found in these species are identical to each other, i.e. sequences found in M. bovis are identical to those found in M. tuberculosis, and so on.

The provision of a diagnostic reagent which is a “MTC diagnostic reagent” indicates that the diagnostic reagent is capable of generating a positive result in a skin test conducted on an animal infected or previously exposed to a MTC species, or in an in vitro assay conducted on a sample obtained from such an animal. A “positive result” is determined in accordance with standard assay protocols, as will be explained further herein in relation to specific tests and assays. A positive result is not observable when the diagnostic reagent is utilised in a test conducted on an animal which is not so infected or previously exposed to, or on a sample obtained from such an animal.

In one embodiment, the MTC diagnostic reagent is a M. bovis, M. tuberculosis, M. africanum, M. orygis, and/or M. caprae diagnostic reagent. Alternatively, the MTC diagnostic reagent may be a M. bovis, M. tuberculosis, M. orygis, and/or M. caprae diagnostic reagent. In another embodiment, the MTC diagnostic reagent is a M. bovis, M. tuberculosis, and/or M. caprae diagnostic reagent. The MTC diagnostic reagent may be a M. africanum, M. orygis, and/or M. caprae diagnostic reagent. In an embodiment, the MTC diagnostic reagent comprises a M. bovis and/or M. tuberculosis diagnostic reagent.

The MTC diagnostic reagent may comprise or consist of a Mycobacterium bovis (M. bovis) and/or Mycobacterium tuberculosis (M. tuberculosis) diagnostic reagent comprising the reagent components:
e) a Rv3616c antigen polypeptide and/or a Rv3616c antigenic cocktail;
f) a Rv1789 antigen polypeptide and/or a Rv1789 antigenic cocktail;
g) a Rv3810 antigen polypeptide and/or a Rv3810 antigenic cocktail; and
h) a Rv3478 antigen polypeptide and/or a Rv3478 antigenic cocktail.

The provision of a diagnostic reagent which is a “M. bovis and/or M. tuberculosis diagnostic reagent” indicates that the diagnostic reagent is capable of generating a positive result in a skin test conducted on an animal infected with or previously exposed to M. bovis and/or M. tuberculosis, or in an in vitro assay conducted on a sample obtained from such an animal. A “positive result” is determined in accordance with standard assay protocols, as will be explained further herein in relation to specific tests and assays. A positive result is not observable when the diagnostic reagent is utilised in a test conducted on an animal which is not so infected or previously exposed to, or on a sample obtained from such an animal.

An “antigenic cocktail” or “antigenic peptide cocktail” as referred to herein provides a mixture of peptides which have overlapping amino acid sequences such that, between them, the peptides encompass substantially the whole length (e.g., at least about 90%) of the equivalent full length protein. For example, the Rv1789 antigenic peptide cocktail comprising SEQ ID NOs:1-48 encompasses the full length sequence SEQ ID NO:183. Any single peptide within one of the cocktails described herein may be referred to as an “antigenic peptide” even if, in isolation away from the other components of the cocktail, it would not have antigenic properties.

An “antigen polypeptide” is a full-length polypeptide of the indicated antigen, or a longer polypeptide comprising the full-length polypeptide of the indicated antigen (for example within a fusion protein), or a portion of the full-length polypeptide comprising a sequence of amino acids which is at least 80% the length of the full-length polypeptide and which has at least 90% sequence identity to the corresponding portion of the full-length polypeptide.

Such a polypeptide is a “functional variant” as referred to herein, provided it is capable of eliciting an equivalent immune response in an animal or in a sample obtained from an animal, as the immune response to the full-length polypeptide. This may be tested, for example, using an interferon gamma release assay (IGRA) as described herein.

For example, an example of a “Rv1789 antigen polypeptide” is SEQ ID NO:183 (see Table 4 below), and a functional variant thereof might comprise SEQ ID NO:183 having up to about 75 amino acids in total removed from the sequence by deletion from the ends, for example having up to about 1, 2, 3, 4, 5, 6, 7, 8, 9, 10, 15, 20, 25 or up to about 30 amino acids deleted from the N- and/or C-terminal. Alternatively, up to about 1, 2, 3, 4, 5, 6, 7, 8, 9, 10, 15, 20, 25 or up to about 30 amino acids may be added to the N- and/or C-terminal of the polypeptide. A functional variant might also comprise an amino acid deletion, addition or substitution at up to about 1, 2, 3, 4, 5, 6, 7, 8, 9, 10, 15, 20, 25 or up to about 30 amino acid positions within the antigen polypeptide amino acid sequence. For example, a “conservative substitution” of one or more amino acids may be permissible, as outlined below.

The diagnostic reagent as described herein is capable of distinguishing between an animal (particularly a mammal such as a bovine animal, a badger or a human being) which is infected with (or has previously been exposed to) a MTC species (particularly a Mycobacterium bovis and/or Mycobacterium tuberculosis bacterium), and an animal which is not so infected or has not been so exposed. In particular, the diagnostic reagent as described herein is advantageously capable of use to detect infection with or exposure to a MTC species, particularly M. bovis and/or M. tuberculosis, even when detection of infection or exposure has not been possible using a DIVA reagent, such as a DIVA reagent comprising ESAT-6, CFP-10 and/or Rv3615c polypeptides or antigenic fragments thereof. Such detection is possible by means of the use of the diagnostic reagent in an IGRA conducted on peripheral blood mononuclear cells (PBMC) obtained from the animal, or by use of the diagnostic reagent as a skin test reagent, in a skin test conducted on the animal. Suitable tests are described elsewhere herein.

The diagnostic reagent according to the first aspect of the invention may further comprise at least one further reagent component selected from:
a) a ESAT-6 antigen polypeptide and/or a ESAT-6 antigenic cocktail;
b) a CFP-10 antigen polypeptide and/or a CFP-10 antigenic cocktail;
c) a Rv3615c antigen polypeptide and/or a Rv3615c antigenic cocktail;
d) SEQ ID NO:207 (a fusion protein of ESAT-6 and CFP-10 and Rv3615c).

In one embodiment, the diagnostic reagent may comprise or consist of the reagent components:
i) a Rv3616c antigen polypeptide (e.g. SEQ ID NO:208 or a functional variant thereof) and/or a Rv3616c antigenic cocktail (e.g. a cocktail comprising SEQ ID NOs:97-144, or comprising SEQ ID NOs:97-143 and 145, or comprising SEQ ID NOs:187-20);
ii) a Rv1789 antigen polypeptide (e.g. SEQ ID NO:183 or a functional variant thereof) and/or a Rv1789 antigenic cocktail (e.g. a cocktail comprising SEQ ID NOs:1-48);
iii) a Rv3810 antigen polypeptide (e.g. SEQ ID NO:186 or a functional variant thereof) and/or a Rv3810 antigenic cocktail (e.g. a cocktail comprising SEQ ID NOs:146-179);
iv) a Rv3478 antigen polypeptide (e.g. SEQ ID NO:185 or a functional variant thereof) and/or a Rv3478 antigenic cocktail (e.g. a cocktail comprising SEQ ID NOs:49-96);
v) a ESAT-6 antigen polypeptide (e.g. SEQ ID NO:180 or a functional variant thereof) and/or a ESAT-6 antigenic cocktail;
vi) a CFP-10 antigen polypeptide (e.g. SEQ ID NO:181 or a functional variant thereof) and/or a CFP-10 antigenic cocktail;
vii) a Rv3615c antigen polypeptide (e.g. SEQ ID NO:182 or a functional variant thereof) and/or a Rv3615c antigenic cocktail; and
viii) a Rv3020c antigen polypeptide (e.g. SEQ ID NO:184 or a functional variant thereof) and/or a Rv3020c antigenic cocktail.

In an embodiment where the diagnostic reagent “consists of” the reagent components (i)-(viii) defined above, this is an indication that no other MTC (for example M. bovis and/or M. tuberculosis) antigenic polypeptides or antigenic fragments thereof are present, or other polypeptides obtainable from a MTC (for example M. bovis or M. tuberculosis) bacterium. The diagnostic reagent may, however, of course comprise other components such as buffers or adjuvants, for example. The invention therefore encompasses a composition comprising a diagnostic reagent according to the first aspect of the invention which consists of the reagent components described, wherein the composition does not further comprise any MTC (for example M. bovis and/or M. tuberculosis) antigenic polypeptides or antigenic fragments thereof, or other polypeptides obtainable from a MTC (for example M. bovis or M. tuberculosis) bacterium. The composition may comprise buffers or adjuvants as described elsewhere herein, or any other non-MTC (e.g. non-M. bovis and/or non-M. tuberculosis) components.

Examples of ESAT-6, CFP-10, Rv3615c antigenic cocktails are described, by way of example, in WO2009/060184, WO2011/135369 and Millington et al. (2011) Proc. Natl. Acad. Sci. U.S.A. 108 5730. Further cocktails are described in co-pending patent applications U.S. 62/832,034 and GB1906193.6. A Rv3020c antigenic cocktail is described, by way of example, in WO2012/010875.

The diagnostic reagent may comprise a reagent component which is a Rv3616c antigenic cocktail, the cocktail comprising:
a) SEQ ID NOs:97-144;
b) SEQ ID NOs:97-143 and 145; or
c) SEQ ID NOs:187-206;

Alternatively or additionally, the diagnostic reagent may comprise a reagent component which is a Rv3616c antigen polypeptide having SEQ ID NO:208, or comprising a functional variant thereof.

The MTC (for example M. bovis and/or M. tuberculosis) diagnostic reagent may comprise a reagent component which is a Rv1789 antigen polypeptide having SEQ ID NO:183 or a functional variant thereof, and/or comprise a reagent component which is a Rv1789 antigenic cocktail comprising SEQ ID NOs:1-48.

The diagnostic reagent may comprise a reagent component which is a Rv3478 antigen polypeptide having SEQ ID NO:185 or a functional variant thereof, and/or comprise a reagent component which is a Rv3478 antigenic cocktail comprising SEQ ID NOs:49-96.

The diagnostic reagent may comprise a reagent component which is a Rv3810 antigen polypeptide having SEQ ID NO:186 or a functional variant thereof, and/or comprise a reagent component which is a Rv3810 antigenic cocktail comprising SEQ ID NOs:146-179.

The diagnostic reagent may comprise a reagent component which is an ESAT-6 antigen polypeptide having SEQ ID NO:180 or a functional variant thereof, and/or comprise a reagent component which is an ESAT-6 antigenic cocktail as described in one or more of WO2009/060184, WO2011/135369 and Millington et al. (2011) Proc. Natl. Acad. Sci. U.S.A. 108 5730.

The diagnostic reagent may comprise a reagent component which is a CFP-10 antigen polypeptide having SEQ ID NO:181 or a functional variant thereof, and/or comprise a reagent component which is a CFP-10 antigenic cocktail as described in one or more of WO2009/060184, WO2011/135369 and Millington et al. (2011) Proc. Natl. Acad. Sci. U.S.A. 108 5730.

The diagnostic reagent may comprise a reagent component which is a Rv3615c antigen polypeptide having SEQ ID NO:182 or a functional variant thereof, and/or comprise a reagent component which is a Rv3615c antigenic cocktail as described in one or more of WO2009/060184, WO2011/135369 and Millington et al. (2011) Proc. Natl. Acad. Sci. U.S.A. 108 5730.

The diagnostic reagent may comprise a reagent component which is an antigenic cocktail of peptides derived from ESAT-6, CFP-10 and Rv1315c, as described in co-pending patent applications US 62/832,034 and GB1906193.6, defined therein as a “skin test diagnostic reagent”.

The diagnostic reagent may comprise a reagent component which is a Rv3020c antigen polypeptide having SEQ ID NO:184 or a functional variant thereof, and/or comprise a reagent component which is a Rv3020c antigenic cocktail as described in WO2012/010875.

In an embodiment, the MTC (for example, M. bovis and/or M. tuberculosis) diagnostic reagent according to the invention comprises or consists of SEQ ID NOs:97-144 and 180-186. Any one or more of these sequences may be replaced by or complemented with a functional variant of the one or more sequences.

In an embodiment, the MTC (for example, M. bovis and/or M. tuberculosis) diagnostic reagent according to the invention comprises or consists of the antigenic peptides having sequences SEQ ID NOs:180-206. Any one or more of these peptides may be replaced by or complemented with a functional variant of the sequence, as explained below.

In an embodiment where the diagnostic reagent “consists of” the reagent components SEQ ID NOs:97-144 and 180-186, or “consists of” the reagent components SEQ ID NOs:180-206, this is an indication that no other MTC (for example, M. bovis and/or M. tuberculosis) antigenic polypeptides or antigenic fragments thereof are present, or other polypeptides obtainable from a MTC (for example, M. bovis or M. tuberculosis) bacterium. The diagnostic reagent may, however, of course comprise other components.

For example, the diagnostic reagent (or the composition referred to above) may comprise one or more adjuvants and/or excipients. However, in some embodiments (particularly if intended for use as a diagnostic reagent in a skin test), the diagnostic reagent does not comprise an adjuvant, i.e., a reagent that assists in propagating an immune response to enhance the effect of the diagnostic reagent, but which does not itself induce an immune response. An example is a bacterial lipopeptide and the skilled person is readily able to determine the identity of a suitable adjuvant in a given context. It may be desirable to avoid the use of adjuvants particularly in a reagent intended for use in a skin test, since repeat skin test injections (as is required to monitor the health of, for example, a herd of dairy cattle) may lead to the sensitisation of non-tuberculosis infected animals, so that the skin test would cease to be useful to differentiate between infected animals and uninfected but vaccinated animals.

The diagnostic reagent (or the composition referred to above) may be in the form (particularly if intended for use as a diagnostic reagent in a skin test) of a sterile injectable preparation which may be an aqueous or an oleaginous suspension, or a suspension in a non-toxic parenterally-acceptable diluent or solvent. The aqueous suspension may be prepared in, for example, mannitol, water, Ringer's solution or isotonic sodium chloride solution. Alternatively, it may be prepared in phosphate buffered saline solution. The oleaginous suspension may be prepared in a synthetic monoglyceride, a synthetic diglyceride, a fatty acid or a natural pharmaceutically-acceptable oil. The fatty acid may be an oleic acid or an oleic acid glyceride derivative. The natural pharmaceutically-acceptable oil may be an olive oil, a castor oil, or a polyoxyethylated olive oil or castor oil. The oleaginous suspension may contain a long-chain alcohol diluent or dispersant, for example, Ph. HeIv.

The diagnostic reagent (or the composition referred to above) may also be in a form comprising a buffer solution (such as a RPMI medium, for example RPMI-1640) which may optionally further comprise DMSO. Such a formulation may be suitable for use in an in vitro assay such as an IGRA as described herein.

In the MTC (for example, M. bovis and/or M. tuberculosis) diagnostic reagent according to the first aspect of the invention, if the reagent (or the composition referred to above) is in liquid form each polypeptide or peptide included within the reagent may be present at a concentration of about 1 μg/ml to about 10 mg/ml. By way of non-limiting example, for preparation as a stock reagent (for example for storage and subsequent dilution prior to use), a liquid diagnostic reagent (or the composition referred to above) according to the invention may comprise each polypeptide or peptide at a concentration of about 10 mg/ml. By way of further non-limiting example, for use in an IGRA a liquid diagnostic reagent according to the invention may comprise each polypeptide or peptide at a concentration of 1-10 μg/ml, for example about 5 μg/ml. By way of further non-limiting example, for use in a skin test a liquid diagnostic reagent (or the composition referred to above) according to the invention may comprise each polypeptide or peptide at a concentration of 0.1-1 mg/ml, for example about 100 μg/ml or about 0.5, 0.6, 0.7 or about 0.8 mg/ml.

The diagnostic reagent (or the composition referred to above) may be in liquid form (including a liquid which is frozen), or may be in dried or lyophilised form. The diagnostic reagent (or the composition referred to above) may be prepared in liquid form comprising each polypeptide or peptide in the concentrations indicated above, followed by a freezing, drying, lyophilising or desiccating process (by way of non-limiting example). Such methods for preparation of reagents into a form suitable for storage are part of the routine ability of the skilled person.

The MTC diagnostic reagent according to the first aspect of the invention may be for use in a method of detecting in an animal infection with or exposure to one or more MTC species, the method comprising contacting the animal with the diagnostic reagent and/or comprising obtaining a biological sample from the animal and contacting the sample with the diagnostic reagent. In particular, the method may be defined in accordance with the second aspect of the invention.

For example, the diagnostic reagent according to the first aspect of the invention may be for use in a method of detecting in an animal infection with or exposure to M. tuberculosis, M. africanum, M. orygis, M. bovis, M. microti, M. canetti, M. caprae, M. pinnipedii, M. suricattae and/or M. mungi.

In embodiments where the diagnostic reagent is a M. bovis and/or M. tuberculosis diagnostic reagent according to the first aspect of the invention, the diagnostic reagent may be for use in a method of detecting in an animal infection with or exposure to M. bovis and/or M. tuberculosis, the method comprising contacting the animal with the diagnostic reagent and/or comprising obtaining a biological sample from the animal and contacting the sample with the diagnostic reagent. In particular, the method may be defined in accordance with the second aspect of the invention.

A second aspect of the invention provides a method of diagnosing in an animal infection with or exposure to one or more Mycobacterium Tuberculosis Complex (MTC) species, the method comprising contacting the animal or a sample obtained therefrom with:
(01) a Rv3616c reagent component comprising a Rv3616c antigen polypeptide (e.g. SEQ ID NO:208 or a functional variant thereof) and/or a Rv3616c antigenic peptide cocktail (e.g. a cocktail comprising SEQ ID NOs:97-144, or comprising SEQ ID NOs:97-143 and 145, or comprising SEQ ID NOs:187-20);
(02) a Rv1789 reagent component comprising a Rv1789 antigen polypeptide (e.g. SEQ ID NO:183 or a functional variant thereof) and/or a Rv1789 antigenic cocktail (e.g. a cocktail comprising SEQ ID NOs:1-48);
(03) a Rv3810 reagent component comprising a Rv3810 antigen polypeptide (e.g. SEQ ID NO:186 or a functional variant thereof) and/or a Rv3810 antigenic cocktail (e.g. a cocktail comprising SEQ ID NOs:146-179); and
(04) a Rv3478 reagent component comprising a Rv3478 antigen polypeptide (e.g. SEQ ID NO:185 or a functional variant thereof) and/or a Rv3478 antigenic cocktail (e.g. a cocktail comprising SEQ ID NOs:49-96).

In an embodiment, the method is a method of diagnosing in an animal infection with or exposure to M. bovis and/or M. tuberculosis, the method comprising contacting the animal or a sample obtained therefrom with:
(01) a Rv3616c reagent component comprising a Rv3616c antigen polypeptide (e.g. SEQ ID NO:208 or a functional variant thereof) and/or a Rv3616c antigenic peptide cocktail (e.g. a cocktail comprising SEQ ID NOs:97-144, or comprising SEQ ID NOs:97-143 and 145, or comprising SEQ ID NOs:187-20);
(02) a Rv1789 reagent component comprising a Rv1789 antigen polypeptide (e.g. SEQ ID NO:183 or a functional variant thereof) and/or a Rv1789 antigenic cocktail (e.g. a cocktail comprising SEQ ID NOs:1-48);
(03) a Rv3810 reagent component comprising a Rv3810 antigen polypeptide (e.g. SEQ ID NO:186 or a functional variant thereof) and/or a Rv3810 antigenic cocktail (e.g. a cocktail comprising SEQ ID NOs:146-179); and
(04) a Rv3478 reagent component comprising a Rv3478 antigen polypeptide (e.g. SEQ ID NO:185 or a functional variant thereof) and/or a Rv3478 antigenic cocktail (e.g. a cocktail comprising SEQ ID NOs:49-96).

The animal may be a mammal, for example a bovine mammal, a badger or a human being. The method may comprise a step of observing an immune response in the animal or the sample obtained therefrom and correlating the presence of the response with the occurrence in the animal or infection by or exposure to one or more MTC species (for example, M. bovis and/or M. tuberculosis) (i.e., the presence of the immune response enables a conclusion to be reached that infection by or exposure to one or more MTC species, such as M. bovis and/or M. tuberculosis has occurred). An immune response may be observed, for example, by means of a positive result in a skin test conducted on the animal as described elsewhere herein, or by means of a positive result in a cytokine release test or a test based on any other blood-derived parameter. A cytokine release test may be any test which measures the amount of a cytokine. A cytokine may be, for example, a chemokine, interferons and/or interleukins. Such tests may be conducted on a whole blood sample obtained from the animal, or from peripheral blood mononuclear cells (PBMCs) obtained from a sample obtained from the animal, as described elsewhere herein. By way of non-limiting example, a cytokine release test may comprise an interferon gamma release assay (IGRA) conducted on a whole blood sample obtained from the animal, or from PBMCs obtained from a sample obtained from the animal. A cytokine release test may also comprise detection of Interleukin-2 (IL2) and/or Interferon gamma-induced protein 10 (IP-10); such tests may also be conducted on a whole blood sample obtained from the animal, or from PBMCs obtained from a sample obtained from the animal.

The Rv3616c, Rv1789, Rv3810 and Rv3478 reagent components (01, 02, 03, 04 above) may be provided as a single combined reagent component which is a MTC (for example M. bovis and/or M. tuberculosis) diagnostic reagent according to the first aspect of the invention.

In an embodiment, the method according to the second aspect of the invention may comprise obtaining a biological sample from the animal and conducting a cytokine release test such as an IGRA, or other cytokine or chemokine release assay, or a test based on any other blood-derived parameter, on the sample using the Rv3616c, Rv1789, Rv3810 and Rv3478 reagent components (described as 01, 02, 03, 04 above). The terms “biological sample” and “sample” are used interchangeably herein to refer to a sample of whole blood or a sample of cells such as PBMCs derived from a whole blood sample which has been obtained from the animal.

Alternatively, the method according to the second aspect of the invention may comprise conducting a skin test on the animal, the skin test comprising administration of the diagnostic reagent to the animal. “Administration” to the animal may comprise intradermal injection of the diagnostic reagent at one or more sites on the skin of the animal. In some embodiments, 10 μg of each polypeptide or antigenic peptide included within the diagnostic reagent may be administered to the animal.

The term “skin test” as referred to herein may be any of a CFT, SIT or SICCT test, as described in the Office International des Epizooties (OIE) Manual of Diagnostic Tests and Vaccines for Terrestrial Animals 2019 (www.oie.int/standard-setting/terrestrial-manual/access-online/, accessed 9 Jul. 2019) in Chapter 3.4.6. The manual provides information, definitions and guidelines on positive test criteria. Therefore, when the MTC (for example, M. bovis and/or M. tuberculosis) diagnostic reagent according to the invention elicits a positive result when administered in a skin test such as one of those mentioned above, this is determined, for example, by detection of an increased thickness and/or induration of skin at the site at which the diagnostic reagent has been injected, using callipers, for example. The skin thickness may ideally be determined, for example, prior to injection (to provide a starting thickness for comparison after injection) and at one or more of, for example, about 24, 36, 48, 72, 96 or about 120 hours after injection of the diagnostic reagent. Determining skin thickness at about 72 hours after injection is typical. Thickness may be determined at any time period after injection, provided that, when results from different tests are compared, they are compared after substantially the same time period after injection (e.g., between 1 and 10 hours before or after one of the time points mentioned above such as the 72 hour time point, for example, between 3 and 7 hours before or after or about 5 hours before or after).

A third aspect of the invention provides a diagnostic kit comprising
(01) a Rv3616c reagent component comprising a Rv3616c antigen polypeptide (e.g. SEQ ID NO:208 or a functional variant thereof) and/or a Rv3616c antigenic peptide cocktail (e.g. a cocktail comprising SEQ ID NOs:97-144, or comprising SEQ ID NOs:97-143 and 145, or comprising SEQ ID NOs:187-20);
(02) a Rv1789 reagent component comprising a Rv1789 antigen polypeptide (e.g. SEQ ID NO:183 or a functional variant thereof) and/or a Rv1789 antigenic cocktail (e.g. a cocktail comprising SEQ ID NOs:1-48);
(03) a Rv3810 reagent component comprising a Rv3810 antigen polypeptide (e.g. SEQ ID NO:186 or a functional variant thereof) and/or a Rv3810 antigenic cocktail (e.g. a cocktail comprising SEQ ID NOs:146-179); and
(04) a Rv3478 reagent component comprising a Rv3478 antigen polypeptide (e.g. SEQ ID NO:185 or a functional variant thereof) and/or a Rv3478 antigenic cocktail (e.g. a cocktail comprising SEQ ID NOs:49-96).

The diagnostic kit may further comprise one or more of the reagent components:
(05) a ESAT-6 antigen polypeptide (e.g. SEQ ID NO:180 or a functional variant thereof) and/or a ESAT-6 antigenic cocktail;
(06) a CFP-10 antigen polypeptide (e.g. SEQ ID NO:181 or a functional variant thereof) and/or a CFP-10 antigenic cocktail;
(07) a Rv3615c antigen polypeptide (e.g. SEQ ID NO:182 or a functional variant thereof) and/or a Rv3615c antigenic cocktail; and
(08) a Rv3020c antigen polypeptide (e.g. SEQ ID NO:184 or a functional variant thereof) and/or a Rv3020c antigenic cocktail.

The Rv3616c, Rv1789, Rv3810 and Rv3478 reagent components (01, 02, 03, 04 above) may be provided in the kit as a single combined reagent component which is a MTC (for example M. bovis and/or M. tuberculosis) diagnostic reagent according to the first aspect of the invention. Such a diagnostic reagent may, as described above in relation to the first aspect of the invention, also comprise one or more or all of the reagent components 05, 06, 07 and/or 08.

The diagnostic kit may, therefore, comprise any diagnostic reagent according to the first aspect of the invention.

The diagnostic kit may further comprise additional components, for example solutions for use to reconstitute any of the reagent components 01-08 which are present (either as individual reagent components, or within a diagnostic reagent (or composition) according to the first aspect of the invention) in the kit in a dried, lyophilised or desiccated form. For example, in the event that the diagnostic kit provides reagents for use in a skin test method, the kit may further comprise a sterile injectable solution which may be useful to reconstitute the reagent components prior to administration in a skin test. In addition, or alternatively, the kit may further comprise apparatus for intradermal administration of the reagent components to at least one site on the skin of an animal to be subjected to a skin test method as described herein. Alternatively, the kit may comprise other reagents necessary for conducting an assay such as an IGRA as described herein.

The diagnostic kit according to the third aspect of the invention may be for use in the method according to the second aspect of the invention. The diagnostic kit may comprise reagent components useable to detect a MTC species infection in an animal. Preferably, the diagnostic kit comprises reagent components useable to detect a M. bovis and/or M. tuberculosis infection in an animal.

Preferably, the reagent components are useable to differentiate between an animal infected with a MTC species and an animal vaccinated against infection by a MTC species, typically by detection of infection or exposure when used in combination or conjunction with a DIVA reagent comprising ESAT-6, CFP-10 and/or Rv3615c polypeptides or antigenic fragments thereof, for example by inclusion in the kit of one or more of reagent components 05, 06, 07 and/or 08 described above.

Preferably, the reagent components are useable to differentiate between an animal infected with M. bovis and/or M. tuberculosis and an animal vaccinated against infection by M. bovis and/or M. tuberculosis, typically by detection of infection or exposure when used in combination or conjunction with a DIVA reagent comprising ESAT-6, CFP-10 and/or Rv3615c polypeptides or antigenic fragments thereof, for example by inclusion in the kit of one or more of reagent components 05, 06, 07 and/or 08 described above.

The present invention also encompasses diagnostic reagent components comprising functional variants of the identified polypeptides and antigenic peptides and methods utilising these variant polypeptides and peptides. For example, the diagnostic reagent according to the invention may further comprise one or more functional variants of the identified polypeptides and peptides. The variant is still functionally active in that it still elicits a positive result when administered in a skin test to an animal infected with one or more MTC species, for example M. bovis or M. tuberculosis, or when utilised in a cytokine release test such as an IGRA conducted on a sample of whole blood obtained from the animal, or on a sample of PBMCs derived from a whole blood sample obtained from the animal, or a test based on any other blood-derived parameter conducted on a biological sample as referred to herein.

As used herein, a “variant” means a polypeptide in which the amino acid sequence differs from the base sequence from which it is derived in that one or more amino acids within the sequence are substituted for other amino acids (or in that one or more amino acids are deleted or added). The variant is a functional variant, in that the functional characteristics of the polypeptide from which the variant is derived are maintained. For example, a similar immune response is elicited by exposure of an animal, or a sample from an animal, to the variant polypeptide as to the non-variant. Specifically, the functional variant still elicits a positive result when administered in a skin test to an animal infected with one or more MTC species, for example, M. bovis or M. tuberculosis, or when utilised in a cytokine release test such as an IGRA conducted on a sample of whole blood obtained from the animal, or on a sample of PBMCs derived from a whole blood sample obtained from the animal, or a test based on any other blood-derived parameter conducted on a biological sample as referred to herein. In particular, any amino acid substitutions, additions or deletions must not alter or significantly alter any tertiary structure of one or more epitopes contained within the polypeptide from which the variant is derived. The skilled person is readily able to determine appropriate functional variants and to determine the tertiary structure of an epitope and any alterations thereof, without the application of inventive skill.

Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

| Class | Amino acid examples |
|---|---|
| Nonpolar: | Ala, Val, Leu, Ile, Pro, Met, Phe, Trp |
| Uncharged polar: | Gly, Ser, Thr, Cys, Tyr, Asn, Gln |
| Acidic: | Asp, Glu |
| Basic: | Lys, Arg, His. |

As is well known to those skilled in the art, altering the primary structure of a polypeptide by a conservative substitution may not significantly alter the activity of that polypeptide because the side-chain of the amino acid which is inserted into the sequence may be able to form similar bonds and contacts as the side chain of the amino acid which has been substituted out. This is so even when the substitution is in a region which is critical in determining the polypeptide's conformation.

As mentioned above, non-conservative substitutions are possible provided that these do not disrupt the tertiary structure of an epitope within the polypeptide, for example, which do not interrupt the immunogenicity (for example, the antigenicity) of the polypeptide.

Broadly speaking, fewer non-conservative substitutions will be possible without altering the biological activity of the polypeptide. Suitably, variants may be at least 80% identical, for example at least 85%, 86%, 87%, 88%, 89%, 90%, 91%, 92%, 93%, 94%, 95%, 96%, 97%, 97.5%, 98% or at least 99% identical to the base sequence.

Sequence identity between amino acid sequences can be determined by comparing an alignment of the sequences. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences, to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Sequence identity preferably is determined using the Needleman-Wunsch Global Sequence Alignment Tool available from the National Center for Biotechnology Information (NCBI), Bethesda, Md., USA, for example via www.blast.ncbi.nlm.nih.gov/Blast.cgi, using default parameter settings.

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires; in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows the interferon gamma release assay (IGRA) results of stimulation of peripheral blood mononuclear cells (PBMCs) with Rv1789, Rv3478, Rv3616c or Rv3810 induced significantly stronger IFN-γ responses in M. bovis infected cattle compared to uninfected controls. Cryo-preserved PBMC from infected and uninfected animals were stimulated for 3 days with these antigens. IFN-γ production was determined in supernatants by Bovigam ELISA. Statistical analysis using t-test, with p-values shown in the Figure.

FIG. 2 shows skin test results obtained by testing M. bovis infected animals (left panel) and uninfected control calves (right panel) using various reagents. Reactions were read at 0 and 72 h and data are expressed as increase in skin reactions at the 72 h read point (Δskin thickness in mm). Horizontal bars: median responses. Statistical analysis by Wilcoxon signed ranks test. ****, P<0.0001, NS, not significant.

FIG. 3 shows a comparison of skin test responses induced by TRT1 and TRT2 in infected and uninfected control animals. Reactions were read at 0 and 72 h and data are expressed as increase in skin reactions at the 72 h read point (Δskin thickness in mm). Horizontal bars: median responses. Statistical analysis by Wilcoxon signed ranks test. ***, P<0.001, NS, not significant.

FIG. 4 shows Receiver Operator Characteristic (ROC) Analysis of TRT1 (left panel) and TRT2 (right panel) performance. Results are summarised in the tables accompanying the figure panels.

FIG. 5 shows a comparison of IGRA responses induced by DST and TRT2 reagents in infected and uninfected control animals. Whole blood from 22 infected and 30 uninfected control calves were stimulated for 20 h with DST and TRT2 reagents at different concentrations. IFN-γ production was quantified by ELISA and expressed as nil antigen corrected values (ΔOD). Symbols and solid horizontal lines represent individual animals and group medians respectively. The dotted horizontal line indicates the preliminary cut-off for positivity (ΔOD>0.1).

FIG. 6 shows skin test results obtained by testing M. bovis experimentally infected (n=20), naturally infected (n=21), uninfected control (n=30) and Gudair-vaccinated (n=29) calves using various reagents. Reactions were read at 0 and 72 h and data are expressed as increase in skin reactions at the 72 h read point (Δskin thickness in mm). Horizontal bars: median responses. Statistical analysis by Friedman test with Dunn's post test. **, p<0.01; ***, p<0.001; ****, p<0.0001.

FIG. 7 shows a comparison of IGRA responses induced by various reagents in M. bovis experimentally infected (n=20), naturally infected (n=21), uninfected control (n=30) and Gudair-vaccinated (n=29) calves. Whole blood from calves were stimulated for 20 h with PPDA, PPDB, B-A (PPD-B-PPD-A; otherwise referred to as the SICCT-test), DST or TRT2 reagents. IFN-γ production was quantified by ELISA and expressed as nil antigen corrected values (ΔOD). Symbols and solid horizontal lines represent individual animals and group medians respectively. The dotted horizontal line indicates the preliminary cut-off for positivity (ΔOD>0.1). Statistical analysis by Friedman test with Dunn's post test. **, p<0.01; ***, P<0.001; ****, p<0.0001.

### DETAILED DESCRIPTION

### Materials and Methods

### Preparation of Antigens

(a) for in vitro assay Antigens are referred to herein in accordance with standard M. tuberculosis nomenclature, since sequences found in M. bovis (as well as M. africanum, M. orygis, M. microti, M. canetti, M. caprae, M. pinnipedii, M. suricattae and M. mungi) are identical to those found in M. tuberculosis. Antigen sequences may be obtained via mycobrowser.epfl.ch (accessed 10 Jul. 2019), searching for sequences from M. tuberculosis H37Rv.

The candidate antigens listed in Table 1, to be screened in the peripheral blood mononuclear (PBMC) assay, were prepared either as as recombinant proteins or as separate pools of overlapping synthetic peptides (20-mers overlapping by 12 amino acids; JPT Peptide Technologies, Germany). Examples of the preparation of peptide pools for various antigens is well understood by the skilled person and is described for certain antigens, for example, in W02009/060184, W02011/135369 and Millington et al. (2011) Proc. Natl. Acad. Sci. U.S.A. 108 5730.

Details of the peptide pools for Rv1789, Rv3478, Rv3616c, and Rv3810 are shown in Table 4 below. The lyophilized peptide pools were reconstituted in RPMI 1640 (Gibco Life Technologies, UK) containing 2.25% DMSO to obtain a concentration of 55 μg of each peptide/ml, with the exception of Rv3616c which was reconstituted in RPMI 1640 containing 25% DMSO to obtain a concentration of 1 mg of each peptide/ml. All peptide pools were used to stimulate cattle PBMC at a final concentration of 5 μg of each peptide/ml. As a control, a recombinant fusion protein consisting of the antigens ESAT-6, CFP-10 and Rv3615c (Rv-EC; Lionex Ltd; SEQ ID NO:207) was used at a final concentration of 5 μg/ml.

(b) for in vivo skin testing ESAT-6, CFP-10, Rv1789, Rv3020c, Rv3478, Rv3615c and Rv3810 were sourced as recombinant proteins from a commercial manufacturer (Lionex Ltd, Germany, sequences shown in Table 5 below).

Rv3616c was prepared as either (i) Rv3616c_{(JPT)}: a peptide pool of 48 synthetic peptides (SEQ ID NOs:97-144, each overlapping by 12 amino acids; JPT Peptide Technologies) where the lyophilized peptide pool was reconstituted in PBS to obtain a concentration of 0.8 mg of each peptide/ml; or (ii) Rv3616c_{(Gen)}: a synthetic peptide pool consisting of sixteen 40-mers, three 25-mers and one 20-mer (SEQ ID NOs:187-206; GenScript Biotech, Netherlands) where each individual lyophilized peptide was first reconstituted in PBS to a concentration of 10 mg/ml and then combined together to obtain a peptide pool of 0.5 mg of each peptide/ml. Details of the peptide sequences included in the pools are shown in Table 5 below.

Skin test reagents TRT1 and TRT2 were then formulated by combining ESAT-6, CFP-10, Rv1789, Rv3020c, Rv3478, Rv3615c and Rv3810 proteins with either Rv3616c_{(JPT)} (TRT1) or Rv3616c_{(Gen)} (TRT2), so that each protein or individual peptide was at a concentration of 100 μg/ml. As a control, a skin test reagent (DST) comprised of ESAT-6, CFP-10 and Rv3615c proteins only was also formulated at 100 μg of each protein/ml. Bovine tuberculin (PPD-B) and avian tuberculin (PPD-A) were obtained from a commercial manufacturer (Thermo Fisher). Information on the sequences included in the reagents is shown in Table 6 below.

### Animals

For the in vitro testing of antigens, archived PBMC from the following groups of cattle (Bos taurus taurus) were used:
(i) naturally M. bovis-infected cattle originating from UK herds known to have bTB (natural infection was confirmed by post-mortem and/or culture analysis);
(ii) non-infected control cattle originating from UK herds in the Low Risk Area that were Officially TB Free for over 5 years; and
(iii) Gudair Johne's disease-vaccinated cattle originating from GB herds that were

Officially TB Free for over 5 years.

For in vivo testing of skin test reagents, the following groups of cattle were used:
(i) experimentally M. bovis-infected cattle consisting of male calves experimentally infected with approx. 10,000 CFU of a field strain of M. bovis (AF2122/97) via the endobronchial route (infection was confirmed by post-mortem and/or culture analysis);
(ii) non-infected control cattle as described above;

1(iii) naturally M. bovis-infected cattle originating from herds from the Republic of Ireland known to have confirmed bTB; and
(iv) Gudair-vaccinated cattle as described above.

The experimentally M. bovis-infected cattle were skin tested 5 weeks post infection. Tuberculin skin test-positive cattle (based on the comparative cervical tuberculin test) were selected from herds with persistent and confirmed bTB as the naturally M. bovis infected cattle. Animal procedures were approved by the APHA Animal Welfare and Ethical Review Board.

### In Vitro Stimulation of PBMC

Cryo-preserved PBMC were thawed as quickly as possible in a water bath at 37° C. Upon thawing, appropriate volume of complete media (RPMI 1640 containing 2 mM GlutaMax, 25 mM HEPES, 0.1 mM NEAA, 5×10⁻⁵M β-mercaptoethanol, 100 U/ml penicillin, 100 μg/ml streptomycin (Gibco Life Technologies, UK) and 10% fetal calf serum (Sigma-Aldrich, UK)) was added in a dropwise manner and centrifuged at 350 g for 10 minutes at room temperature. The supernatant was discarded, the cell pellet gently loosened and resuspended in complete media and the cells counted using a hemocytometer. PBMC were plated at 2×10⁵ cells/well in 96-well plates and stimulated with and without antigens for 3 days at 37° C. in the presence of 5% CO₂, following which cell supernatants were removed and stored at −80° C. until required.

### IFN-γ ELISA

Quantification of IFN-γ in PBMC culture supernatant was determined using the commercially available BOVIGAM enzyme-linked immunosorbent assay (ELISA) kit (Thermo Fisher Scientific, USA). Results were expressed as the optical density at 450 nm (OD₄₅₀) for cultures stimulated with antigen minus the OD450 for cultures without antigen (i.e. ΔOD₄₅₀).

### Skin Test Procedure

Injection sites located in the border of the anterior and middle third of the neck on either side of the cow were clipped and skin thickness recorded. PPD-A and PPD-B were administered in a 0.1 ml volume via intradermal injection as per manufacturer's recommendations. DST, TRT1 and TRT2 reagents were administered in a similar manner so that each individual protein or peptide was delivered at a 10 μg dose. To account for potential injection site differences, a Latin Square design was applied with animals randomly assigned to the Latin Square combinations. Skin thickness was measured again by the same operator 72 hours after administration, and the difference in skin thickness (mm) between the pre- and post-skin test readings recorded.

### Statistical Analysis

All statistical analyses were performed using Prism 7 (Graphpad Software, USA).

### Results

### Example 1

### Production of Candidate Antigens

Eighteen candidate proteins were selected for testing (see Table 1). These proteins were sourced from commercial sources, when available, as recombinant proteins. However, for the majority of proteins, this was not possible. In these cases, overlapping synthetic peptide sets were designed and commercially produced using state-of-the-art high-throughput peptide synthesis chemistry.

### Antigen Screening and Complementation

These antigens were screened in interferon gamma release assays (IGRA) using bio-banked peripheral blood mononuclear cells (PBMC) from previous experiments and projects. Samples were obtained from naturally infected field reactors as well as uninfected controls.

To down-select to the most promising candidate antigens, the following gating criteria were applied:
Significantly stronger IGRA responses induced by antigens in PBMC from M. bovis infected animals compared to uninfected controls;
Specificity (i.e. no responses in uninfected animals including animals with high avian PPD responses);
Antigens complement responses to the existing DIVA skin test antigens (ESAT-6, CFP-10, and Rv3615c).

Four antigens fulfilled all three of these criteria: Rv1789, Rv3478, Rv3616c, Rv3810 (italicised in Table 1). This was surprising since several other candidate antigens had been identified previously as being potentially useful in the development of diagnostic reagents (Cockle et al. (2006) Clin. Vaccine Immunol. 13 1119; Jones et al. (2010) Infect. Immun. 78 1326; Jones et al. (2010) Clin. Vaccine Immunol. 17 1344; Jones et al. (2013) Clin. Vaccine Immunol. 20 1675; Mustafa et al. Infect. Immun. 74 4566).

TABLE 1
Summary of responses to candidate antigens.

| | Significant IFN-γ: | | |
|---|---|---|---|
| | Infected > Controls | Specific | Complementation |
| Rv1789 | Yes | Yes | Yes |
| Rv3478 | Yes | Yes | Yes |
| Rv3616c | Yes | Yes | Yes |
| Rv3810 | Yes | Yes | Yes |
| Rv0288 | No | N/A | No |
| Rv0445c | No | N/A | No |
| Rv1038c | No | N/A | No |
| Rv1195 | No | N/A | No |
| Rv1197 | No | N/A | No |
| Rv1253 | No | N/A | No |
| Rv1387 | No | N/A | No |
| Rv1792 | No | N/A | No |
| Rv1983 | No | N/A | No |
| Rv2608 | No | N/A | No |
| Rv3017c | No | N/A | No |
| Rv3444c | No | N/A | No |
| Rv3872 | No | N/A | No |
| Rv3783 | No | N/A | No |

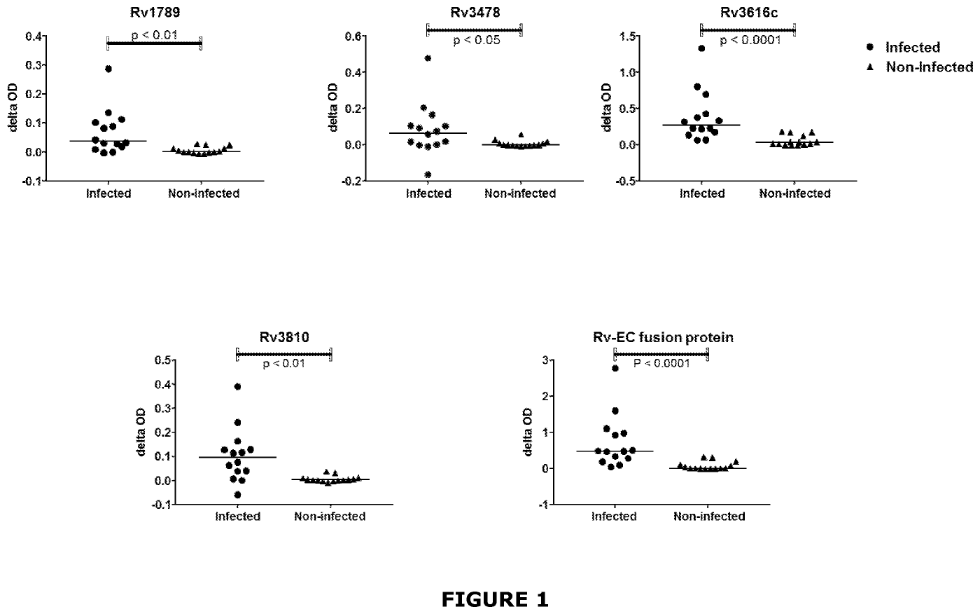

The IGRA responses for these four antigens are shown in FIG. 1 in comparison with responses induced by the DIVA skin test fusion protein (SEQ ID NO:207, a fusion of Rv3615c, ESAT6, CFP10). Responses to the peptide pools derived from each of the four proteins were significantly higher in PBMC from infected compared to uninfected cattle (p-values<0.0001 to 0.05), with no significant responses induced in T cells from uninfected animals.

PBMC from seven infected cattle did not respond to ESAT-6 (data not shown). The peptide pool for each of the four antigens described above were recognised by between one and five of these animals demonstrating their potential to complement the DIVA skin test antigens to increase overall signal strength and sensitivity (data not shown). This observation was confirmed when we considered three animals not recognising the DIVA skin test fusion protein, one of which responded to the Rv3616c peptide pool.

Based on these results, these four antigens were selected for in vivo assessment. This newly formed ‘TRT’ skin test cocktail included the three DST antigens (ESAT-6, CFP-10, Rv3615c), the four antigens listed above (Rv1789, Rv3478, Rv3616c, Rv3810) as well as an additional antigen (Rv3020c) that we had hitherto identified to induce specific immune responses in infected animals, but lacked the high specificity in BCG vaccinated calves which is a requirement for antigens to be used in a DIVA reagent (Jones et al. (2010) Clin. Vaccine Immunol. 17 1344; Jones et al. (2012) Clin. Vaccine Immunol. 19 620).

### Example 2

### Testing TRT Reagents In Vivo by Skin Testing

Most of the antigens were produced as recombinant proteins by Lionex GmbH (Braunschweig, Germany). Only the antigen Rv3616c could not be produced as a recombinant protein as it proved to be lytic to hosts utilised for expression. To overcome this technical problem, a set of 48 overlapping synthetic peptides was produced (SEQ ID NOs: 97-144). The TRT1 cocktail (see Table 6) was formed by mixing this Rv3616c overlapping peptide set with the protein antigens (Rv1789, Rv3478, Rv3810, ESAT-6, CFP-10, Rv3615c and Rv3020c).

We also designed and procured a second Rv3616c cocktail of 20 peptides, composed of 40-, 25- and 20-mer peptides (SEQ ID NOs:187-206) which were combined with the recombinant proteins described above into TRT2 (see Table 6).

We infected 42 calves via the endobronchial route with around 10,000 CFU M. bovis AF2122/97. Six weeks later, skin tests were performed using PPD-A, PPD-B, DST and TRT1. Injection sites were assigned in individual animals with a Latin Square design with animals randomly assigned to the different sub-groups in the Latin Square applying the double lottery principle. Infection was confirmed at necroscopy by the presence of visible pathology and M. bovis culture. To test for specificity a set of uninfected control calves was skin tested with PPD-B (n=30), PPD-A (n=30), DST (n=30), TRT1 (n=20) and TRT2 (n=30).

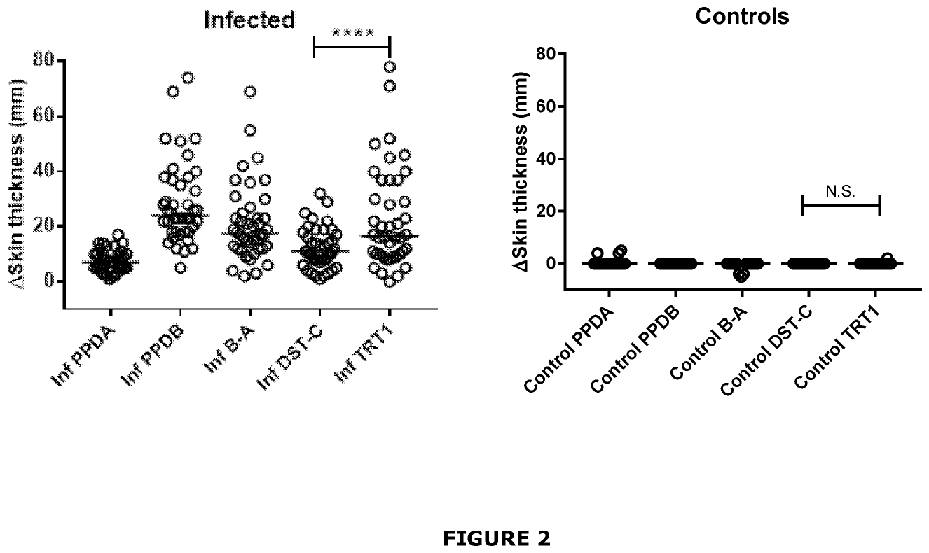

As shown in FIG. 2, statistically highly significantly stronger responses were induced in infected calves following injection of TRT1 compared to DST injection (P<0.0001). In contrast, only 1/20 of the uninfected controls gave rise to a small response (2 mm) with TRT1, whilst none of the DST induced responses were above 1 mm (FIG. 2).

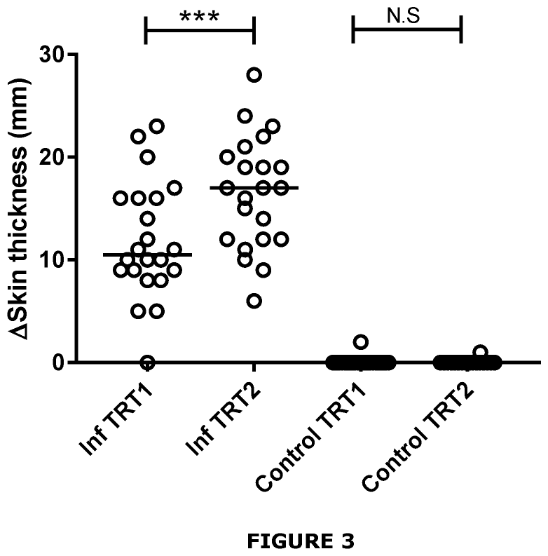

In a subset of animals (n=22), the TRT2 reagent was tested alongside the other skin test reagents described above. We compared responses induced by TRT2 with those induced with TRT1 in a subset of infected animals as well as in uninfected controls (FIG. 3).

The results presented in FIG. 3 clearly demonstrate that TRT2 induced a significantly stronger skin response in infected cattle compared to TRT1 (P<0.001), whilst not inducing skin test responses above 1 mm (1/30) in uninfected controls (FIG. 3). Together, the data presented in FIGS. 2 and 3 therefore clearly demonstrate that the addition of additional antigens to the DST protein cocktail can significantly increase the strength of reactivity without compromising specificity. Furthermore, TRT2, which contains fewer but longer peptides representing Rv3616c, is a markedly improved formulation.

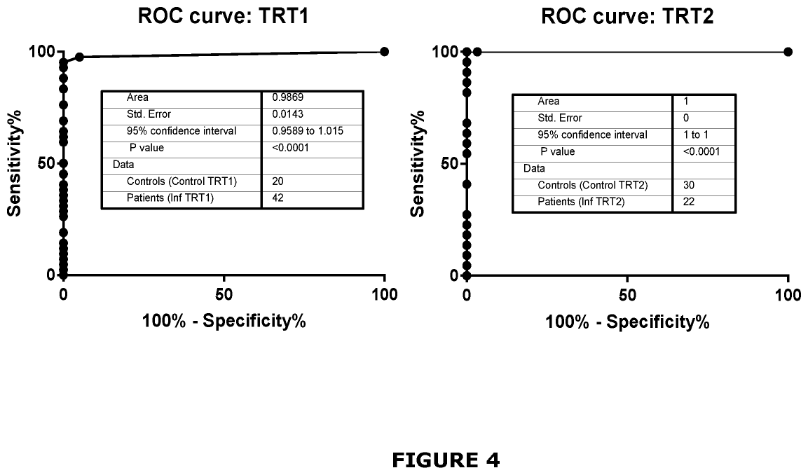

The raw data presented in FIGS. 2 and 3 was used to undertake ROC (Receiver Operating Characteristic) analysis with a view to define the diagnostic performance of TRT1 and TRT2, to define cut-offs for positivity and to estimate their relative sensitivities when we set specificity at 100%. These ROC analyses are shown in FIG. 4 and Table 2. As FIG. 4 shows, both TRT1 and TRT2 are high performance diagnostic antigens when applied in skin tests. This could be demonstrated by the high areas under the curves (TRT1: 0.9869 (95% CI: 0.9589, 1.0); TRT2: 1 (95% CI: 1, 1); P<0.0001 for both analyses).

TABLE 2
Definition of provisional TRT1 and TRT2 cut-off values at 100%
specificity.

| | | | Sensitivity % (95% CI) |
|---|---|---|---|
| Antigen | Specificity % | Cut-off | n/N |
| SICCT, | 100% | >4 mm | 93% (81, 98) |
| standard | | | 39/42 |
| SICCT, | 100% | >2 mm | 98% (88, 100) |
| severe | | | 41/42 |
| SIT | 100% | 4 mm and | 100% (88, 100) |
| | | greater | 42/42 |
| DST | 100% | 2 mm and | 98% (88, 100) |
| | | greater | 41/42 |
| TRT1 | 100% | 3 mm and | 95% (84, 99) |
| | | greater | 40/42 |
| TRT2 | 100% | 5 mm and | 100% (85, 100) |
| | | greater | 22/22 |

We further investigated the outcome of the ROC analyses by assessing relative sensitivity in the test animals after setting test specificity at 100% (Table 2). This allowed us to define cut-points for TRT1 and TRT2 positivity in accordance with routine methods. Compared to the DST cut-off of 2 mm and higher set in previous studies, the cut-off points for TRT1 and TRT2 were 3 mm and higher and 5 mm and higher respectively. These cut-offs maintained high sensitivity values (95% and 100% for TRT1 and TRT2 respectively), comparable to SICCT at standard or severe interpretation, SIT or DST sensitivities (Table 2). Thus, we have achieved the objective of demonstrating significantly stronger skin test responses with TRT1 and TRT2 compared to the DST, thus being able to define higher cut-off values, which lead to a more robust test in terms of reading the skin test results (since the cut-off can be adjusted to increase test sensitivity).

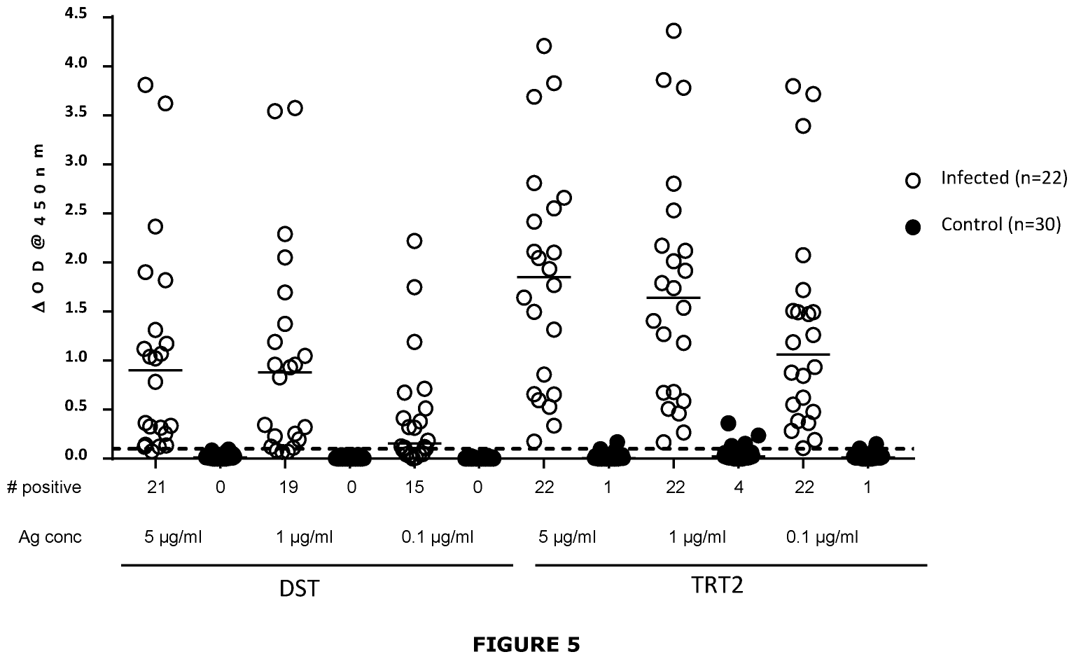

The TRT2 cocktail was also tested for its diagnostic potential in whole blood IGRA assays. To this end, we performed antigen dose titration experiments for both DST and TRT2 reagents using whole blood from experimentally M. bovis infected (n=22) and uninfected control (n=30) calves (FIG. 5). Using the standard PPD-6 minus PPD-A assay readout, all 22 infected calves tested positive, whereas 2 of the 30 control calves tested positive (data not shown). Using a preliminary cut-off (ΔO.D>0.1), 21 out of 22 infected animals tested positive to the DST reagent at the highest concentration tested (5 μg/ml), which decreased to 19 and 15 test positives at the lower antigen concentrations (1 μg/ml and 0.1 μg/ml respectively). In contrast, all infected animals tested positive to the TRT2 reagent at all antigen concentrations. Indeed, a significantly greater proportion of the infected calves tested positive at the lowest concentration of the TRT2 reagent compared to the DST (P-value=0.0089, Fisher's exact test). None of the control animals gave a positive response to the DST reagent at any of the concentrations tested. At the 1 μg/ml TRT2 concentration used four calves tested positive to the TRT2 reagent. Interestingly, one of these was also an animal that gave a positive PPD-6 minus PPD-A result. However, at the lowest antigen concentration tested (0.1 μg/ml), only one control animal remained positive to the TRT2 reagent, whilst responses were maintained in the infected animals. These results highlight the potential of the TRT2 antigens for use in IGRA as an ancillary test to the skin test, using the provisionally defined dose of 0.1 μg/ml with a cut-off value of ΔOD>0.1; although antigen dosage and appropriate cut-offs may still need to be fully defined and possibly refined using data from future studies.

### Example 3

### Testing TRT Reagents in Other Settings

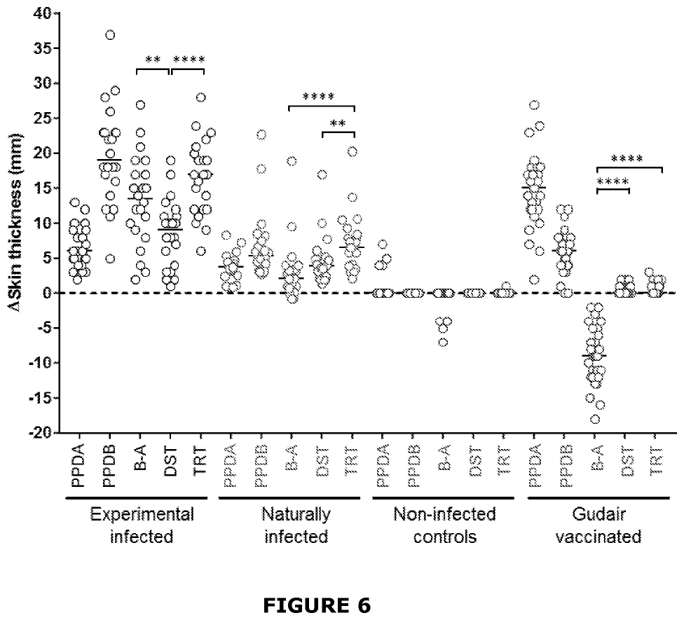

We then investigated the TRT2 cocktail in other in vivo and in vitro animal categories. The other categories were naturally infected cattle, which is more reflective of a field situation, and in animals strongly sensitised to M. a. sso paratuberculosis antigens, which was achieved by vaccinating calves with the Gudair vaccine. These were compared to experimentally infected cattle and non-infected controls, as described above. Skin tests were performed using PPD-A, PPD-B, DST and TRT2. These results are shown in FIG. 6, including B-A, which represents the results of PPD-6 minus PPD-A (SICCT test). As shown in FIG. 6, statistically significantly stronger responses were induced in naturally infected calves following injection of TRT2 compared to DST injection (P<0.01). In contrast, no or low responses were observed in the Gudair-vaccinated calves. Only 3/29 of the vaccinated calves gave rise to a small (2 mm) response to TRT2. Only 1/29 of the vaccinated calves gave rise to a small (3 mm) response to TRT2. No response was observed from the remaining 25/29 vaccinated calves to TRT2 (FIG. 6). The level of reactivity to TRT2 in the Gudair-vaccinated calves was marginally higher than in naive, unvaccinated calves (FIG. 6). These data further demonstrate that the TRT protein cocktail can significantly increase the strength of reactivity as compared to DST, without compromising specificity.

The higher responses to TRT2 in naturally infected animals compared to the DST, allowed us to re-appraise the cut-off for positivity for the TRT in accordance with routine methods. By applying a TRT2 cut-off of 4 mm and higher, we could achieve the same level of specificity as seen in naïve animals (Table 3), whilst being comparable to the sensitivity achieved with PPD-B alone (SIT, Table 3). Thus, we have shown the significantly stronger skin test response with TRT2 compared to the DST. This has allowed us to define higher cut-off values, which lead to a more accurate interpretation of the skin test.

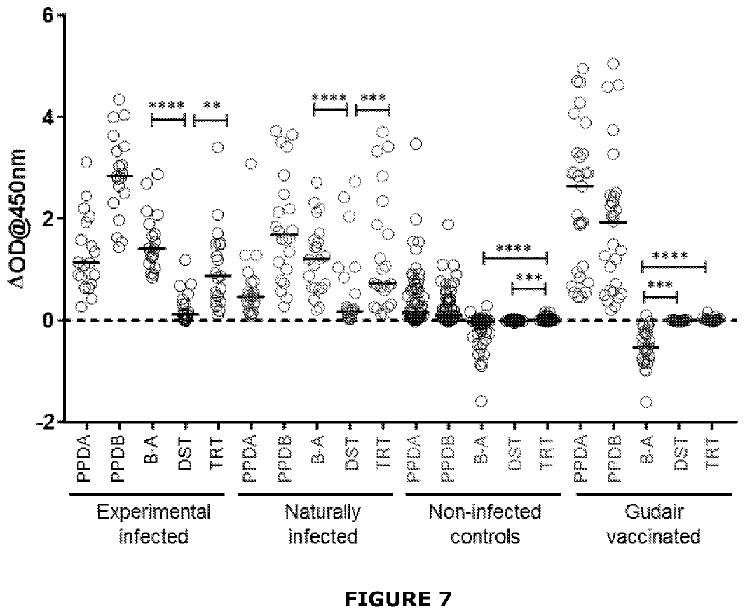

The in vitro diagnostic potential of the TRT2 cocktail for naturally infected and vaccinated cattle was also tested using whole blood IGRA assays. Antigen exposure experiments were performed for PPD-A, PPD-B, DST and TRT2 reagents using whole blood from naturally infected cattle and from the Gudair-vaccinated animals. The data of these experiments are shown in FIG. 7 (together with B-A, i.e. PPD-B-PPD-A). As with the skin test, TRT2 induced significantly stronger IGRA responses than the DST in the naturally infected animals (P<0.001). In contrast, the IGRA response to TRT2 in samples from Gudair-vaccinated animals was minimal or absent below the cut-off for positivity (FIG. 7, cut-off used: OD450 with antigen minus OD without antigen>0.1). These results therefore suggest that the TRT will have utility as an auxiliary test to be used alongside skin testing. These results further confirm the potential of the TRT2 antigens for use in IGRA as an ancillary test to the skin test.

TABLE 3
Comparison of skin test results at defined cut off values.

| | Naturally infected | | Controls | | Gudair vaccinated | |
|---|---|---|---|---|---|---|
| Cut off | DST-C | TRT-2 | DST-C | TRT-2 | DST-C | TRT-2 |
| >=2 mm | 81 [60, 92] | 100 [85, 100] | 0 [0, 11] | 0 [0, 11] | 10 [4, 26] | 14 [5, 31] |
| | (17/21) | (21/21) | (0/30) | (0/30) | (3/29) | (4/29) |
| >=3 mm | 71 [50, 86] | 95 [77, 100] | 0 [0, 11] | 0 [0, 11] | 0 [0, 12] | 3 [0, 17] |
| | (15/21) | (20/21) | (0/30) | (0/30) | (0/29) | (1/29) |
| >=4 mm | 48 [28, 68] | 76 [55, 89]* | 0 [0, 11] | 0 [0, 11] | 0 [0, 12] | 0 [0, 12] |
| | (10/21) | (16/21) | (0/30) | (0/30) | (0/29) | (0/29) |
| >=5 mm | 29 [14, 50] | 71 [50, 86]** | 0 [0, 11] | 0 [0, 11] | 0 [0, 12] | 0 [0, 12] |
| | (6/21) | (15/21) | (0/30) | (0/30) | (0/29) | (0/29) |

| | | | |
|---|---|---|---|
| SIT | 76 [55, 89] | 0 [0, 11] | 79 [62, 90] |
| | (16/21) | (0/30) | (23/29) |
| SICCT | 14 [5, 35] | 0 [0, 11] | 0 [0, 12] |
| (Std) | (3/21) | (0/30) | (0/29) |
| SICCT | 52 [32, 72] | 0 [0, 11] | 0 [0, 12] |
| (Sv) | (11/21) | (0/30) | (0/29) |

*p < 0.05,
**p < 0.01 McNemar test (compared to DST-C).

TABLE 4
Peptide pool details used for in vitro PBMC screening.

| | | | | |
|---|---|---|---|---|
| | Mtb | | | SEQ ID |
| Antigen | designation | Name | Amino acid sequence | NO. |

Rv1789 antigenic peptide pool

| | | | | |
|---|---|---|---|---|
| PPE26 | Rv1789 | Peptide_001 | MDFGALPPEVNSVRMYAGPG | 1 |
| PPE26 | Rv1789 | Peptide_002 | EVNSVRMYAGPGSAPMVAAA | 2 |
| PPE26 | Rv1789 | Peptide_003 | AGPGSAPMVAAASAWNGLAA | 3 |
| PPE26 | Rv1789 | Peptide_004 | VAAASAWNGLAAELSSAATG | 4 |
| PPE26 | Rv1789 | Peptide_005 | GLAAELSSAATGYETVITQL | 5 |
| PPE26 | Rv1789 | Peptide_006 | AATGYETVITQLSSEGWLGP | 6 |
| PPE26 | Rv1789 | Peptide_007 | ITQLSSEGWLGPASAAMAEA | 7 |
| PPE26 | Rv1789 | Peptide_008 | WLGPASAAMAEAVAPYVAWM | 8 |
| PPE26 | Rv1789 | Peptide_009 | MAEAVAPYVAWMSAAAAQAE | 9 |
| PPE26 | Rv1789 | Peptide_010 | VAWMSAAAAQAEQAATQARA | 10 |
| PPE26 | Rv1789 | Peptide_011 | AQAEQAATQARAAAAAFEAA | 11 |
| PPE26 | Rv1789 | Peptide_012 | QARAAAAAFEAAFAATVPPP | 12 |
| PPE26 | Rv1789 | Peptide_013 | FEAAFAATVPPPLIAANRAS | 13 |
| PPE26 | Rv1789 | Peptide_014 | VPPPLIAANRASLMQLISTN | 14 |
| PPE26 | Rv1789 | Peptide_015 | NRASLMQLISTNVFGQNTSA | 15 |
| PPE26 | Rv1789 | Peptide_016 | ISTNVFGQNTSAIAAAEAQY | 16 |
| PPE26 | Rv1789 | Peptide_017 | NTSAIAAAEAQYGEMWAQDS | 17 |
| PPE26 | Rv1789 | Peptide_018 | EAQYGEMWAQDSAAMYAYAG | 18 |
| PPE26 | Rv1789 | Peptide_019 | AQDSAAMYAYAGSSASASAV | 19 |
| PPE26 | Rv1789 | Peptide_020 | AYAGSSASASAVTPFSTPPQ | 20 |
| PPE26 | Rv1789 | Peptide_021 | ASAVTPFSTPPQIANPTAQG | 21 |
| PPE26 | Rv1789 | Peptide_022 | TPPQIANPTAQGTQAAAVAT | 22 |
| PPE26 | Rv1789 | Peptide_023 | TAQGTQAAAVATAAGTAQST | 23 |
| PPE26 | Rv1789 | Peptide_024 | AVATAAGTAQSTLTEMITGL | 24 |
| PPE26 | Rv1789 | Peptide_025 | AQSTLTEMITGLPNALQSLT | 25 |
| PPE26 | Rv1789 | Peptide_026 | ITGLPNALQSLTSPLLQSSN | 26 |
| PPE26 | Rv1789 | Peptide_027 | QSLTSPLLQSSNGPLSWLWQ | 27 |
| PPE26 | Rv1789 | Peptide_028 | QSSNGPLSWLWQILFGTPNF | 28 |
| PPE26 | Rv1789 | Peptide_029 | WLWQILFGTPNFPTSISALL | 29 |
| PPE26 | Rv1789 | Peptide_030 | TPNFPTSISALLTDLQPYAS | 30 |
| PPE26 | Rv1789 | Peptide_031 | SALLTDLQPYASFFYNTEGL | 31 |
| PPE26 | Rv1789 | Peptide_032 | PYASFFYNTEGLPYFSIGMG | 32 |
| PPE26 | Rv1789 | Peptide_033 | TEGLPYFSIGMGNNFIQAAK | 33 |
| PPE26 | Rv1789 | Peptide_034 | IGMGNNFIQAAKTLGLIGSA | 34 |
| PPE26 | Rv1789 | Peptide_035 | QAAKTLGLIGSAAPAAVAAA | 35 |
| PPE26 | Rv1789 | Peptide_036 | IGSAAPAAVAAAGDAAKGLP | 36 |
| PPE26 | Rv1789 | Peptide_037 | VAAAGDAAKGLPGLGGMLGG | 37 |
| PPE26 | Rv1789 | Peptide_038 | KGLPGLGGMLGGGPVAAGLG | 38 |
| PPE26 | Rv1789 | Peptide_039 | MLGGGPVAAGLGNAASVGKL | 39 |
| PPE26 | Rv1789 | Peptide_040 | AGLGNAASVGKLSVPPVWSG | 40 |
| PPE26 | Rv1789 | Peptide_041 | VGKLSVPPVWSGPLPGSVTP | 41 |
| PPE26 | Rv1789 | Peptide_042 | VWSGPLPGSVTPGAAPLPVS | 42 |
| PPE26 | Rv1789 | Peptide_043 | SVTPGAAPLPVSTVSAAPEA | 43 |
| PPE26 | Rv1789 | Peptide_044 | LPVSTVSAAPEAAPGSLLGG | 44 |
| PPE26 | Rv1789 | Peptide_045 | APEAAPGSLLGGLPLAGAGG | 45 |
| PPE26 | Rv1789 | Peptide_046 | LLGGLPLAGAGGAGAGPRYG | 46 |
| PPE26 | Rv1789 | Peptide_047 | GAGGAGAGPRYGFRPTVMAR | 47 |
| PPE26 | Rv1789 | Peptide_048 | GAGPRYGFRPTVMARPPFAG | 48 |

Rv3478 antigenic peptide pool

| | | | | |
|---|---|---|---|---|
| PPE60 | Rv3478 | Peptide_001 | VVDFGALPPEINSARMYAGP | 49 |
| PPE60 | Rv3478 | Peptide_002 | PEINSARMYAGPGSASLVAA | 50 |
| PPE60 | Rv3478 | Peptide_003 | YAGPGSASLVAAAKMWDSVA | 51 |
| PPE60 | Rv3478 | Peptide_004 | LVAAAKMWDSVASDLFSAAS | 52 |
| PPE60 | Rv3478 | Peptide_005 | DSVASDLFSAASAFQSVVWG | 53 |
| PPE60 | Rv3478 | Peptide_006 | SAASAFQSVVWGLTVGSWIG | 54 |
| PPE60 | Rv3478 | Peptide_007 | VVWGLTVGSWIGSSAGLMAA | 55 |
| PPE60 | Rv3478 | Peptide_008 | SWIGSSAGLMAAAASPYVAW | 56 |
| PPE60 | Rv3478 | Peptide_009 | LMAAAASPYVAWMSVTAGQA | 57 |
| PPE60 | Rv3478 | Peptide_010 | YVAWMSVTAGQAQLTAAQVR | 58 |
| PPE60 | Rv3478 | Peptide_011 | AGQAQLTAAQVRVAAAAYET | 59 |
| PPE60 | Rv3478 | Peptide_012 | AQVRVAAAAYETAYRLTVPP | 60 |
| PPE60 | Rv3478 | Peptide_013 | AYETAYRLTVPPPVIAENRT | 61 |
| PPE60 | Rv3478 | Peptide_014 | TVPPPVIAENRTELMTLTAT | 62 |
| PPE60 | Rv3478 | Peptide_015 | ENRTELMTLTATNLLGQNTP | 63 |
| PPE60 | Rv3478 | Peptide_016 | LTATNLLGQNTPAIEANQAA | 64 |
| PPE60 | Rv3478 | Peptide_017 | QNTPAIEANQAAYSQMWGQD | 65 |
| PPE60 | Rv3478 | Peptide_018 | NQAAYSQMWGQDAEAMYGYA | 66 |
| PPE60 | Rv3478 | Peptide_019 | WGQDAEAMYGYAATAATATE | 67 |
| PPE60 | Rv3478 | Peptide_020 | YGYAATAATATEALLPFEDA | 68 |
| PPE60 | Rv3478 | Peptide_021 | TATEALLPFEDAPLITNPGG | 69 |
| PPE60 | Rv3478 | Peptide_022 | FEDAPLITNPGGLLEQAVAV | 70 |
| PPE60 | Rv3478 | Peptide_023 | NPGGLLEQAVAVEEAIDTAA | 71 |
| PPE60 | Rv3478 | Peptide_024 | AVAVEEAIDTAAANQLMNNV | 72 |
| PPE60 | Rv3478 | Peptide_025 | DTAAANQLMNNVPQALQQLA | 73 |
| PPE60 | Rv3478 | Peptide_026 | MNNVPQALQQLAQPAQGVVP | 74 |
| PPE60 | Rv3478 | Peptide_027 | QQLAQPAQGVVPSSKLGGLW | 75 |
| PPE60 | Rv3478 | Peptide_028 | GVVPSSKLGGLWTAVSPHLS | 76 |
| PPE60 | Rv3478 | Peptide_029 | GGLWTAVSPHLSPLSNVSSI | 77 |
| PPE60 | Rv3478 | Peptide_030 | PHLSPLSNVSSIANNHMSMM | 78 |
| PPE60 | Rv3478 | Peptide_031 | VSSIANNHMSMMGTGVSMTN | 79 |
| PPE60 | Rv3478 | Peptide_032 | MSMMGTGVSMTNTLHSMLKG | 80 |
| PPE60 | Rv3478 | Peptide_033 | SMTNTLHSMLKGLAPAAAQA | 81 |
| PPE60 | Rv3478 | Peptide_034 | MLKGLAPAAAQAVETAAENG | 82 |
| PPE60 | Rv3478 | Peptide_035 | AAQAVETAAENGVWAMSSLG | 83 |
| PPE60 | Rv3478 | Peptide_036 | AENGVWAMSSLGSQLGSSLG | 84 |
| PPE60 | Rv3478 | Peptide_037 | SSLGSQLGSSLGSSGLGAGV | 85 |
| PPE60 | Rv3478 | Peptide_038 | SSLGSSGLGAGVAANLGRAA | 86 |
| PPE60 | Rv3478 | Peptide_039 | GAGVAANLGRAASVGSLSVP | 87 |
| PPE60 | Rv3478 | Peptide_040 | GRAASVGSLSVPPAWAAANQ | 88 |
| PPE60 | Rv3478 | Peptide_041 | LSVPPAWAAANQAVTPAARA | 89 |
| PPE60 | Rv3478 | Peptide_042 | AANQAVTPAARALPLTSLTS | 90 |
| PPE60 | Rv3478 | Peptide_043 | AARALPLTSLTSAAQTAPGH | 91 |
| PPE60 | Rv3478 | Peptide_044 | SLTSAAQTAPGHMLGGLPLG | 92 |
| PPE60 | Rv3478 | Peptide_045 | APGHMLGGLPLGHSVNAGSG | 93 |
| PPE60 | Rv3478 | Peptide_046 | LPLGHSVNAGSGINNALRVP | 94 |
| PPE60 | Rv3478 | Peptide_047 | AGSGINNALRVPARAYAIPR | 95 |
| PPE60 | Rv3478 | Peptide_048 | NNALRVPARAYAIPRTPAAG | 96 |

Rv3616c antigenic peptide pool

| | | | | |
|---|---|---|---|---|
| EspA | Rv3616c | Peptide_001 | MSRAFIIDPTISAIDGLYDL | 97 |
| EspA | Rv3616c | Peptide_002 | PTISAIDGLYDLLGIGIPNQ | 98 |
| EspA | Rv3616c | Peptide_003 | LYDLLGIGIPNQGGILYSSL | 99 |
| EspA | Rv3616c | Peptide_004 | IPNQGGILYSSLEYFEKALE | 100 |
| EspA | Rv3616c | Peptide_005 | YSSLEYFEKALEELAAAFPG | 101 |
| EspA | Rv3616c | Peptide_006 | KALEELAAAFPGDGWLGSAA | 102 |
| EspA | Rv3616c | Peptide_007 | AFPGDGWLGSAADKYAGKNR | 103 |
| EspA | Rv3616c | Peptide_008 | GSAADKYAGKNRNHVNFFQE | 104 |
| EspA | Rv3616c | Peptide_009 | GKNRNHVNFFQELADLDRQL | 105 |
| EspA | Rv3616c | Peptide_010 | FFQELADLDRQLISLIHDQA | 106 |
| EspA | Rv3616c | Peptide_011 | DRQLISLIHDQANAVQTTRD | 107 |
| EspA | Rv3616c | Peptide_012 | HDQANAVQTTRDILEGAKKG | 108 |
| EspA | Rv3616c | Peptide_013 | TTRDILEGAKKGLEFVRPVA | 109 |
| EspA | Rv3616c | Peptide_014 | AKKGLEFVRPVAVDLTYIPV | 110 |
| EspA | Rv3616c | Peptide_015 | RPVAVDLTYIPVVGHALSAA | 111 |
| EspA | Rv3616c | Peptide_016 | YIPVVGHALSAAFQAPFCAG | 112 |
| EspA | Rv3616c | Peptide_017 | LSAAFQAPFCAGAMAVVGGA | 113 |
| EspA | Rv3616c | Peptide_018 | FCAGAMAVVGGALAYLAVKT | 114 |
| EspA | Rv3616c | Peptide_019 | VGGALAYLAVKTLINATQLL | 115 |
| EspA | Rv3616c | Peptide_020 | AVKTLINATQLLKLLAKLAE | 116 |
| EspA | Rv3616c | Peptide_021 | TQLLKLLAKLAELVAAAIAD | 117 |
| EspA | Rv3616c | Peptide_022 | KLAELVAAAIADIISDVADI | 118 |
| EspA | Rv3616c | Peptide_023 | AIADIISDVADIIKGILGEV | 119 |
| EspA | Rv3616c | Peptide_024 | VADIIKGILGEVWEFITNAL | 120 |
| EspA | Rv3616c | Peptide_025 | LGEVWEFITNALNGLKELWD | 121 |
| EspA | Rv3616c | Peptide_026 | TNALNGLKELWDKLTGWVTG | 122 |
| EspA | Rv3616c | Peptide_027 | ELWDKLTGWVTGLFSRGWSN | 123 |
| EspA | Rv3616c | Peptide_028 | WVTGLFSRGWSNLESFFAGV | 124 |
| EspA | Rv3616c | Peptide_029 | GWSNLESFFAGVPGLTGATS | 125 |
| EspA | Rv3616c | Peptide_030 | FAGVPGLTGATSGLSQVTGL | 126 |
| EspA | Rv3616c | Peptide_031 | GATSGLSQVTGLFGAAGLSA | 127 |
| EspA | Rv3616c | Peptide_032 | VTGLFGAAGLSASSGLAHAD | 128 |
| EspA | Rv3616c | Peptide_033 | GLSASSGLAHADSLASSASL | 129 |
| EspA | Rv3616c | Peptide_034 | AHADSLASSASLPALAGIGG | 130 |
| EspA | Rv3616c | Peptide_035 | SASLPALAGIGGGSGFGGLP | 131 |
| EspA | Rv3616c | Peptide_036 | GIGGGSGFGGLPSLAQVHAA | 132 |
| EspA | Rv3616c | Peptide_037 | GGLPSLAQVHAASTRQALRP | 133 |
| EspA | Rv3616c | Peptide_038 | VHAASTRQALRPRADGPVGA | 134 |
| EspA | Rv3616c | Peptide_039 | ALRPRADGPVGAAAEQVGGQ | 135 |
| EspA | Rv3616c | Peptide_040 | PVGAAAEQVGGQSQLVSAQG | 136 |
| EspA | Rv3616c | Peptide_041 | VGGQSQLVSAQGSQGMGGPV | 137 |
| EspA | Rv3616c | Peptide_042 | SAQGSQGMGGPVGMGGMHPS | 138 |
| EspA | Rv3616c | Peptide_043 | GGPVGMGGMHPSSGASKGTT | 139 |
| EspA | Rv3616c | Peptide_044 | MHPSSGASKGTTTKKYSEGA | 140 |
| EspA | Rv3616c | Peptide_045 | KGTTTKKYSEGAAAGTEDAE | 141 |
| EspA | Rv3616c | Peptide_046 | SEGAAAGTEDAERAPVEADA | 142 |
| EspA | Rv3616c | Peptide_047 | EDAERAPVEADAGGGQKVLV | 143 |
| EspA | Rv3616c | Peptide_048 | RAPVEADAGGGQKVLVRNVV | 145 |

Rv3810 antigenic peptide pool

| | | | | |
|---|---|---|---|---|
| PirG | Rv3810 | Peptide_001 | VPNRRRRKLSTAMSAVAALA | 146 |
| PirG | Rv3810 | Peptide_002 | LSTAMSAVAALAVASPCAYF | 147 |
| PirG | Rv3810 | Peptide_003 | AALAVASPCAYFLVYESTET | 148 |
| PirG | Rv3810 | Peptide_004 | CAYFLVYESTETTERPEHHE | 149 |
| PirG | Rv3810 | Peptide_005 | STETTERPEHHEFKQAAVLT | 150 |
| PirG | Rv3810 | Peptide_006 | EHHEFKQAAVLTDLPGELMS | 151 |
| PirG | Rv3810 | Peptide_007 | AVLTDLPGELMSALSQGLSQ | 152 |
| PirG | Rv3810 | Peptide_008 | ELMSALSQGLSQFGINIPPV | 153 |
| PirG | Rv3810 | Peptide_009 | GLSQFGINIPPVPSLTGSGD | 154 |
| PirG | Rv3810 | Peptide_010 | IPPVPSLTGSGDASTGLTGP | 155 |
| PirG | Rv3810 | Peptide_011 | GSGDASTGLTGPGLTSPGLT | 156 |
| PirG | Rv3810 | Peptide_012 | LTGPGLTSPGLTSPGLTSPG | 157 |
| PirG | Rv3810 | Peptide_013 | PGLTSPGLTSPGLTDPALTS | 158 |
| PirG | Rv3810 | Peptide_014 | TSPGLTDPALTSPGLTPTLP | 159 |
| PirG | Rv3810 | Peptide_015 | ALTSPGLTPTLPGSLAAPGT | 160 |
| PirG | Rv3810 | Peptide_016 | PTLPGSLAAPGTTLAPTPGV | 161 |
| PirG | Rv3810 | Peptide_017 | APGTTLAPTPGVGANPALTN | 162 |
| PirG | Rv3810 | Peptide_018 | TPGVGANPALTNPALTSPTG | 163 |
| PirG | Rv3810 | Peptide_019 | ALTNPALTSPTGATPGLTSP | 164 |
| PirG | Rv3810 | Peptide_020 | SPTGATPGLTSPTGLDPALG | 165 |
| PirG | Rv3810 | Peptide_021 | LTSPTGLDPALGGANEIPIT | 166 |
| PirG | Rv3810 | Peptide_022 | PALGGANEIPITTPVGLDPG | 167 |
| PirG | Rv3810 | Peptide_023 | IPITTPVGLDPGADGTYPIL | 168 |
| PirG | Rv3810 | Peptide_024 | LDPGADGTYPILGDPTLGTI | 169 |
| PirG | Rv3810 | Peptide_025 | YPILGDPTLGTIPSSPATTS | 170 |
| PirG | Rv3810 | Peptide_026 | LGTIPSSPATTSTGGGGLVN | 171 |
| PirG | Rv3810 | Peptide_027 | ATTSTGGGGLVNDVMQVANE | 172 |
| PirG | Rv3810 | Peptide_028 | GLVNDVMQVANELGASQAID | 173 |
| PirG | Rv3810 | Peptide_029 | VANELGASQAIDLLKGVLMP | 174 |
| PirG | Rv3810 | Peptide_030 | QAIDLLKGVLMPSIMQAVQN | 175 |
| PirG | Rv3810 | Peptide_031 | VLMPSIMQAVQNGGAAAPAA | 176 |
| PirG | Rv3810 | Peptide_032 | AVQNGGAAAPAASPPVPPIP | 177 |
| PirG | Rv3810 | Peptide_033 | APAASPPVPPIPAAAAVPPT | 178 |
| PirG | Rv3810 | Peptide_034 | PPIPAAAAVPPTDPITVPVA | 179 |

TABLE 5
Additional peptide and protein amino acid sequences.

| | | | | SEQ |
|---|---|---|---|---|
| | Mtb | Peptide/ | | ID |
| Antigen | designation | protein | Amino acid sequence | NO. |
| EspA | Rv3616c | Peptide | EADAGGGQKVLVRNVV | 144 |
| ESAT-6 | Rv3875 | Protein | MTEQQWNFAGIEAAASAIQGNVTSIHSLLDEGKQSLTKLAAAWGGSGSEAYQGVQQKWDATATEL | 180 |
| | | | NNALQNLARTISEAGQAMASTEGNVTGMFA | |
| CFP-10 | Rv3874 | Protein | MAEMKTDAATLAQEAGNFERISGDLKTQIDQVESTAGSLQGQWRGAAGTAAQAAVVRFQEAANKQ | 181 |
| | | | KQELDEISTNIRQAGVQYSRADEEQQQALSSQMGF | |
| EspC | Rv3615c | Protein | MTENLTVQPERLGVLASHHDNAAVDASSGVEAAAGLGESVAITHGPYCSQFNDTLNVYLTAHNALGS | 182 |
| | | | SLHTAGVDLAKSLRIAAKIYSEADEAWRKAIDGLFT | |
| PPE26 | Rv1789 | Protein | MDFGALPPEVNSVRMYAGPGSAPMVAAASAWNGLAAELSSAATGYETVITQLSSEGWLGPASAAMA | 183 |
| | | | EAVAPYVAWMSAAAAQAEQAATQARAAAAAFEAAFAATVPPPLIAANRASLMQLISTNVFGQNTSAIA | |
| | | | AAEAQYGEMWAQDSAAMYAYAGSSASASAVTPFSTPPQIANPTAQGTQAAAVATAAGTAQSTLTEM | |
| | | | ITGLPNALQSLTSPLLQSSNGPLSWLWQILFGTPNFPTSISALLTDLQPYASFFYNTEGLPYFSIGMG | |
| | | | NNFIQSAKTLGLIGSAAPAAVAAAGDAAKGLPGLGGMLGGGPVAAGLGNAASVGKLSVPPVWSGPLP | |
| | | | GSVTPGAAPLPVSTVSAAPEAAPGSLLGGLPLAGAGGAGAGPRYGFRPTVMARPPFAG | |
| EsxS | Rv3020c | Protein | MSLLDAHIPQLIASHTAFAAKAGLMRHTIGQAEQQAMSAQAFHQGESAAAFQGAHARFVAAAAKVN | 184 |
| | | | TLLDIAQANLGEAAGTYVAADAAAASSYTGF | |
| PPE60 | Rv3478 | Protein | VVDFGALPPEINSARMYAGPGSASLVAAAKMWDSVASDLFSAASAFQSVVWGLTVGSWIGSSAGLM | 185 |
| | | | AAAASPYVAWMSVTAGQAQLTAAQVRVAAAAYETAYRLTVPPPVIAENRTELMTLTATNLLGQNTPAI | |
| | | | EANQAAYSQMWGQDAEAMYGYAATAATATEALLPFEDAPLITNPGGLLEQAVAVEEAIDTAAANQLM | |
| | | | NNVPQALQQLAQPAQGVVPSSKLGGLWTAVSPHLSPLSNVSSIANNHMSMMGTGVSMTNTLHSML | |
| | | | KGLAPAAAQAVETAAENGVWAMSSLGSQLGSSLGSSGLGAGVAANLGRAASVGSLSVPPAWAAAN | |
| | | | QAVTPAARALPLTSLTSAAQTAPGHMLGGLPLGHSVNAGSGINNALRVPARAYAIPRTPAAG | |
| PirG | Rv3810 | Protein | VPNRRRRKLSTAMSAVAALAVASPCAYFLVYESTETTERPEHHEFKQAAVLTDLPGELMSALSQGLSQ | 186 |
| | | | FGINIPPVPSLTGSGDASTGLTGPGLTSPGLTSPGLTSPGLTDPALTSPGLTPTLPGSLAAPGTTLAP | |
| | | | TPGVGANPALTNPALTSPTGATPGLTSPTGLDPALGGANEIPITTPVGLDPGADGTYPILGDPTLGTI | |
| | | | PSSPATTSTGGGGLVNDVMQVANELGASQAIDLLKGVLMPSIMQAVQNGGAAAPAASPPVPPIPAAAA | |
| | | | VPPTDPITVPVA | |
| EspA | Rv3616c | Peptide | MSRAFIIDPTISAIDGLYDLLGIGIPNQGGILYSSLEYFE | 187 |
| EspA | Rv3616c | Peptide | LGIGIPNQGGILYSSLEYFEKALEELAAAFPGDGWLGSAA | 188 |
| EspA | Rv3616c | Peptide | KALEELAAAFPGDGWLGSAADKYAGKNRNHVNFFQELADL | 189 |
| EspA | Rv3616c | Peptide | DKYAGKNRNHVNFFQELADLDRQLISLIHDQANAVQTTRD | 190 |
| EspA | Rv3616c | Peptide | DRQLISLIHDQANAVQTTRDILEGAKKGLEFVRPVAVDLT | 191 |
| EspA | Rv3616c | Peptide | ILEGAKKGLEFVRPVAVDLTYIPVVGHALSAAFQAPFCAG | 192 |
| EspA | Rv3616c | Peptide | YIPVVGHALSAAFQAPFCAGAMAVVGGALAYLVVKTLINA | 193 |
| EspA | Rv3616c | Peptide | IISDVADIIKGTLGEVWEFITNALNGLKELWDKLTGWVTG | 194 |
| EspA | Rv3616c | Peptide | TNALNGLKELWDKLTGWVTGLFSRGWSNLESFFAGVPGLT | 195 |
| EspA | Rv3616c | Peptide | GATSGLSQVTGLFGAAGLSASSGLAHADSLASSASLPALA | 196 |
| EspA | Rv3616c | Peptide | SSGLAHADSLASSASLPALAGIGGGSGFGGLPSLAQVHAA | 197 |
| EspA | Rv3616c | Peptide | GIGGGSGFGGLPSLAQVHAASTRQALRPRADGPVGAAAEQ | 198 |
| EspA | Rv3616c | Peptide | STRQALRPRADGPVGAAAEQVGGQSQLVSAQGSQGMGGPV | 199 |
| EspA | Rv3616c | Peptide | VGGQSQLVSAQGSQGMGGPVGMGGMHPSSGASKGTTTKKY | 200 |
| EspA | Rv3616c | Peptide | GMGGMHPSSGASKGTTTKKYSEGAAAGTEDAERAPVEADA | 201 |
| EspA | Rv3616c | Peptide | KGTTTKKYSEGAAAGTEDAERAPVEADAGGGQKVLVRNVV | 202 |
| EspA | Rv3616c | Peptide | AMAVVGGALAYLVVKTLINATQLLK | 203 |
| EspA | Rv3616c | Peptide | VKTLINATQLLKLLAKLAELVAAAI | 204 |
| EspA | Rv3616c | Peptide | LAKLAELVAAAIADIISDVADIIKG | 205 |
| EspA | Rv3616c | Peptide | ESFAGVPGLTGATSGLSQVT | 206 |
| Fusion | Rv3615c/ | Protein | MTMITPSLRRDIHMHHHHHHSMDTENLTVQPERLGVLASHHDNAAVDASSGVEAAAGLGESVAITH | 207 |
| protein* | ESAT-6/ | | GPYCSQFNDTLNVYLTAHNALGSSLHTAGVDLAKSLRIAAKIYSEADEAWRKAIDGLFTHMTEQQWN | |
| | CFP-10 | | FAGIEAAASAIQGNVTSIHSLLDEGKQSLTKLAAAWGGSGSEAYQGVQQKWDATATELNNALQNLA | |
| | | | RTISEAGQAMASTEGNVTGMFAGGMAEMKTDAATLAQEAGNFERISGDLKTQIDQVESTAGS | |
| | | | LQGQWRGAAGTAAQAAVVRFQEAANKQKQELDEISTNIRQAGVQYSRADEEQQQALSSQ | |
| | | | MGFHHHHHH | |
| EspA | Rv3616c | Protein | MSRAFIIDPTISAIDGLYDLLGIGIPNQGGILYSSLEYFEKALEELAAAFPGDGWLGSAADKYAGKNR | 208 |
| | | | NHVNFFQELADLDRQLISLIHDQANAVQTTRDILEGAKKGLEFVRPVAVDLTYIPVVGHALSAAFQAP | |
| | | | FCAGAMAVVGGALAYLVVKTLINATQLLKLLAKLAELVAAAIADIISDVADIIKGTLGEVWEFITNAL | |
| | | | NGLKELWDKLTGWVTGLFSRGWSNLESFFAGVPGLTGATSGLSQVTGLFGAAGLSASSGLAHADSLAS | |
| | | | SASLPALAGIGGGSGFGGLPSLAQVHAASTRQALRPRADGPVGAAAEQVGGQSQLVSAQGSQGMGGP | |
| | | | VGMGGMHPSSGASKGTTTKKYSEGAAAGTEDAERAPVEADAGGGQKVLVRNVV | |

*In the fusion protein, underlined amino acids are from Rv3615c, italicised amino acids are from ESAT-6 and bold amino acids are from CFP-10

TABLE 6
Content of TRT1, TRT2 and DST reagents

| | | Mtb | Included in | |
|---|---|---|---|---|
| Reagent | Antigen | designation | reagent as: | SEQ ID NOs: |
| TRT1 | EspA | Rv3616c | Rv3616c_{(JPT)} | 97-144 |
| | | | antigenic peptide | |
| | | | pool | |
| | ESAT-6 | Rv3875 | Protein | 180 |
| | CFP-10 | Rv3874 | Protein | 181 |
| | EspC | Rv3615c | Protein | 182 |
| | PPE26 | Rv1789 | Protein | 183 |
| | EsxS | Rv3020c | Protein | 184 |
| | PPE60 | Rv3478 | Protein | 185 |
| | PirG | Rv3810 | Protein | 186 |
| TRT2 | EspA | Rv3616c | Rv3616c_{(Gen)} | 187-206 |
| | | | antigenic peptide | |
| | | | pool | |
| | ESAT-6 | Rv3875 | Protein | 180 |
| | CFP-10 | Rv3874 | Protein | 181 |
| | EspC | Rv3615c | Protein | 182 |
| | PPE26 | Rv1789 | Protein | 183 |
| | EsxS | Rv3020c | Protein | 184 |
| | PPE60 | Rv3478 | Protein | 185 |
| | PirG | Rv3810 | Protein | 186 |
| DST | ESAT-6 | Rv3875 | Protein | 180 |
| | CFP-10 | Rv3874 | Protein | 181 |
| | EspC | Rv3615c | Protein | 182 |

1. A Mycobacterium Tuberculosis Complex (MTC) diagnostic reagent comprising the reagent components:
a. a Rv3616c antigen polypeptide and/or a Rv3616c antigenic cocktail;
b. a Rv1789 antigen polypeptide and/or a Rv1789 antigenic cocktail;
c. a Rv3810 antigen polypeptide and/or a Rv3810 antigenic cocktail; and
d. a Rv3478 antigen polypeptide and/or a Rv3478 antigenic cocktail.

2. The MTC diagnostic reagent according to claim 1, wherein the diagnostic reagent comprises a Mycobacterium bovis (M. bovis) and/or Mycobacterium tuberculosis (M. tuberculosis) diagnostic reagent comprising the reagent components:
a. a Rv3616c antigen polypeptide and/or a Rv3616c antigenic cocktail;
b. a Rv1789 antigen polypeptide and/or a Rv1789 antigenic cocktail;
c. a Rv3810 antigen polypeptide and/or a Rv3810 antigenic cocktail; and
d. a Rv3478 antigen polypeptide and/or a Rv3478 antigenic cocktail.

3. The MTC diagnostic reagent according to claim 1 or claim 2 further comprising at least one further reagent component selected from:
a. a ESAT-6 antigen polypeptide and/or a ESAT-6 antigenic cocktail;
b. a CFP-10 antigen polypeptide and/or a CFP-10 antigenic cocktail;
c. a Rv3615c antigen polypeptide and/or a Rv3615c antigenic cocktail;
d. SEQ ID NO:207.

4. The MTC diagnostic reagent according to any one of claims 1 to 3 comprising the reagent components:
a. a Rv3616c antigen polypeptide and/or a Rv3616c antigenic cocktail;
b. a Rv1789 antigen polypeptide and/or a Rv1789 antigenic cocktail;
c. a Rv3810 antigen polypeptide and/or a Rv3810 antigenic cocktail;
d. a Rv3478 antigen polypeptide and/or a Rv3478 antigenic cocktail;
e. a ESAT-6 antigen polypeptide and/or a ESAT-6 antigenic cocktail;
f. a CFP-10 antigen polypeptide and/or a CFP-10 antigenic cocktail;
g. a Rv3615c antigen polypeptide and/or a Rv3615c antigenic cocktail; and
h. a Rv3020c antigen polypeptide and/or a Rv3020c antigenic cocktail.

5. The MTC diagnostic reagent according to any preceding claim comprising a reagent component which is a Rv3616c antigenic cocktail, the cocktail comprising:
a. SEQ ID NOs:97-144;
b. SEQ ID NOs:97-143 and 145; or
c. SEQ ID NOs:187-206;
or comprising a reagent component which is a Rv3616c antigen polypeptide having SEQ ID NO:208.

6. The MTC diagnostic reagent according to any preceding claim comprising a reagent component which is a Rv1789 antigen polypeptide having SEQ ID NO:183 or a functional variant thereof, or comprising a reagent component which is a Rv1789 antigenic cocktail comprising SEQ ID NOs:1-48.

7. The MTC diagnostic reagent according to any preceding claim comprising a reagent component which is a Rv3478 antigen polypeptide having SEQ ID NO:185 or a functional variant thereof, or comprising a reagent component which is a Rv3478 antigenic cocktail comprising SEQ ID NOs:49-96.

8. The MTC diagnostic reagent according to any preceding claim comprising a reagent component which is a Rv3810 antigen polypeptide having SEQ ID NO:186 or a functional variant thereof, or comprising a reagent component which is a Rv3810 antigenic cocktail comprising SEQ ID NOs:146-179.

9. The MTC diagnostic reagent according to any preceding claim comprising a reagent component which is an ESAT-6 antigen polypeptide having SEQ ID NO:180 or a functional variant thereof.

10. The MTC diagnostic reagent according to any preceding claim comprising a reagent component which is a CFP-10 antigen polypeptide having SEQ ID NO:181 or a functional variant thereof.

11. The MTC diagnostic reagent according to any preceding claim comprising a reagent component which is a Rv3615c antigen polypeptide having SEQ ID NO:182 or a functional variant thereof.

12. The MTC diagnostic reagent according to any preceding claim comprising a reagent component which is a Rv3020c antigen polypeptide having SEQ ID NO:184 or a functional variant thereof.

13. The MTC diagnostic reagent according to any preceding claim comprising SEQ ID NOs:97-144 and 180-186 and/or a functional variant of any of these in its place.

14. The MTC diagnostic reagent according to any of claims 1-12 comprising SEQ ID NOs:180-206 and/or a functional variant of any of these in its place.

15. The MTC diagnostic reagent according to any preceding claim for use in a method of detecting in an animal infection with or exposure to one or more MTC species comprising contacting the animal with the diagnostic reagent and/or comprising obtaining a sample from the animal and contacting the sample with the diagnostic reagent.

16. The MTC diagnostic reagent according to claim 15, wherein the diagnostic reagent is an M. bovis and/or M. tuberculosis diagnostic reagent for use in a method of detecting in an animal infection with or exposure to M. bovis and/or M. tuberculosis.

17. A method of diagnosing in an animal infection with or exposure to one or more Mycobacterium Tuberculosis Complex (MTC) species, the method comprising contacting the animal or a sample obtained therefrom with:
a. a Rv3616c reagent component comprising a Rv3616c antigen polypeptide and/or a Rv3616c antigenic peptide cocktail;
b. a Rv1789 reagent component comprising a Rv1789 antigen polypeptide and/or a Rv1789 antigenic cocktail;
c. a Rv3810 reagent component comprising a Rv3810 antigen polypeptide and/or a Rv3810 antigenic cocktail; and
d. a Rv3478 reagent component comprising a Rv3478 antigen polypeptide and/or a Rv3478 antigenic cocktail.

18. The method according to claim 17, wherein the method is a method of diagnosing in an animal infection with or exposure to M. bovis and/or M. tuberculosis.

19. The method according to claim 17 or claim 18 wherein the Rv3616c, Rv1789, Rv3810 and Rv3478 reagent components are in the form of a diagnostic reagent according to any of claims 1-14.

20. The method according to any one of claims 17 to 19 comprising obtaining a biological sample from the animal and conducting a blood-derived parameter release test on the sample using the Rv3616c, Rv1789, Rv3810 and Rv3478 reagent components.

21. The method according to claim 20 wherein the blood-derived parameter release test is a cytokine release test.

22. The method according to claim 21 wherein the cytokine release test is an interferon gamma release assay (IGRA).

23. The method according to claim 19 comprising conducting a skin test on the animal, the skin test comprising administration of the diagnostic reagent to the animal.

24. A diagnostic kit comprising
a. a Rv3616c reagent component comprising a Rv3616c antigen polypeptide and/or a Rv3616c antigenic peptide cocktail;
b. a Rv1789 reagent component comprising a Rv1789 antigen polypeptide and/or a Rv1789 antigenic cocktail;
c. a Rv3810 reagent component comprising a Rv3810 antigen polypeptide and/or a Rv3810 antigenic cocktail; and
d. a Rv3478 reagent component comprising a Rv3478 antigen polypeptide and/or a Rv3478 antigenic cocktail.

25. The diagnostic kit according to claim 24 comprising a diagnostic reagent which comprises the Rv3616c, Rv1789, Rv3810 and Rv3478 reagent components.

26. The diagnostic kit according to claim 24 or 25 for use in the method according to any of claims 17-23.

27. A diagnostic kit according to any of claims 24-26, wherein the diagnostic reagent is able to detect a Mycobacterium Tuberculosis Complex (MTC) species infection in an animal.

28. A diagnostic kit according to any of claims 24-27, wherein the diagnostic reagent is able to detect a M. bovis or M. tuberculosis infection in an animal.

29. A diagnostic kit according to claim 27, wherein the diagnostic reagent is able to differentiate between a MTC species infected animal and an animal vaccinated against a MTC species infection.

30. A diagnostic kit according to claim 28, wherein the diagnostic reagent is able to differentiate between a M. bovis or M. tuberculosis infected animal and an animal vaccinated against M. bovis or M. tuberculosis infection.
